(19) United States
(12) Patent Application Publication
(10) Pub. No.: US 2023/0100243 A1
(43) Pub. Date: Mar. 30, 2023

(54) PEPTIDOMIMETICS AND METHOD OF SYNTHESIS THEREOF

(71) Applicant: Adel NEFZI, Miami, FL (US)

(72) Inventor: Adel NEFZI, Miami, FL (US)

(73) Assignee: The Florida International University Board of Trustees, Miami, FL (US)

(21) Appl. No.: 17/462,594

(22) Filed: Aug. 31, 2021

(51) Int. Cl. C07K 7/08; C07K 7/06

(52) U.S. Cl. CPC C07K 7/08; C07K 7/06

(57) ABSTRACT

The subject invention provides compounds, peptidomimetics, and methods of synthesis thereof. The subject invention provides the synthesis and use of guanidino acids and/or poly guanidino acids not only as vehicles for drug delivery but as toolbox for drug discovery. The peptidomimetic of the subject invention comprises oligo(guanidino acid)s or poly(guanidino acid)s with guanidines as peptide bond surrogates. The incorporation of the guanidine as amide bond surrogates offers significant differences in polarity, hydrogen bonding capability, and acid-base character.

### BACKGROUND OF INVENTION

Typical cancer chemotherapies suffer from side effects associated with, for example, toxicity to normal cells and drug resistance. Coupling a drug to large molecules that are not substrates of efflux pumps and that exhibit excellent intracellular entry, such as cell-penetrating peptides (CPP), could address this problem. However, most cell-penetrating molecules are positively charged lipophilic compounds that have limited diffusion through a tumor's extracellular environment. Thus, a drug or drug conjugate that is not a substrate of efflux pumps and offers high intracellular availability through efficient diffusion through the tumor extracellular matrix (ECM) for fast transmembrane entry is desirable.

Polyarginine peptides are known as one of the widely used classes of CPPs and are used as cellular delivery tools. The presence of the guanidine group in the side chain of arginine was demonstrated to play a key role for the improved ability of arginine-rich peptides to cross the cell membrane. They can transport various bioactive cargos inside cells including nucleic acids, large proteins, and other chemical compounds.

Coupling the drug to cell-penetrating oligoarginine allows efficient cellular entry of the drug-peptide conjugate, which is not a substrate for an efflux pump. Despite the promising outcomes, the unshielded positive charges of drug-conjugates remain an issue for clinical translation. Complexation or coupling to negatively charged glycosaminoglycans, hyaluronic acid or chondroitin sulfate, addresses in vivo circulation issues of positively charged complexes. However, poor and inefficient intracellular entry of these nanomedicines remains a major issue that lowers the overall therapeutic efficacy.

Peptidomimetics are compounds having a protein-like chain designed to mimic a natural peptide or protein, which retain the ability to interact with the biological target and produce the same biological effect. Immense efforts have been directed at improving the pharmacological properties of biologically active peptides by incorporating amino acid and peptide mimetics. These peptide analogues are usually characterized by improved enzymatic stability, bioavailability, and duration of action.

Peptidomimetics can be produced by the modification of existing peptides, or by designing similar systems that mimic peptides. Various synthetic strategies have been developed over the years in order to modulate the conformational flexibility and the peptide character of peptidomimetic compounds. The alteration of peptides to peptidomimetics encompasses side-chain manipulation, turn-mimics, amino acid extension, and backbone modifications. For example, the chemical modifications involve the restriction of conformations performed by the incorporation of conformationally restricted building blocks, such as unnatural amino acids and dipeptide surrogates.

Peptidomimetics are designed to advantageously adjust the molecular properties, and to circumvent some of the problems associated with a natural peptide: e.g., stability against proteolysis and poor bioavailability. Certain other properties, such as receptor selectivity or potency, often can be substantially improved. Thus, peptidemimics have great potentials in drug discovery.

Therefore, there is a need for the design, and synthesis of nature-like peptidomimetics and their modified derivatives for use as drugs or drug carriers to enhance tumor targeting and cellular entry of therapeutics. The design and synthesis of peptidomimetics are important because of the dominant position peptide and protein-protein interactions play in molecular recognition and signaling, especially in living systems

### BRIEF SUMMARY

The subject invention provides compounds, peptidomimetics, and methods of synthesis thereof. The subject invention provides an unexplored field in peptidomimetic chemistry, the synthesis and use of guanidino acids and/or poly guanidino acids in drug discovery, not only as vehicles for drug delivery but also as a toolbox for drug discovery.

In one embodiment, the subject invention provides a peptidomimetic comprising oligo(amino acid)s or poly(amino acid)s, wherein the amino acid residues in the oligo(amino acid)s or poly(amino acid)s are connected with guanidines as peptide bond surrogates.

The incorporation of the guanidine as amide bond surrogates offers significant differences in polarity, hydrogen bonding capability, and acid-base character. The introduction of such modifications to the peptide backbone offers a new class of peptidomimetics that can address peptide and existing peptidomimetics limitations by displaying improved bioavailability, metabolic stability, duration of action, enhanced receptor affinity and selectivity and, especially, improved cell penetrating potential. This novel class of compounds opens a vast range of opportunities associated with intracellular and tissue targets protected by other barriers (e.g., BBB, ocular, lung, skin, and aural).

In one embodiment, the subject invention provides a peptidomimetic having a general structure of:

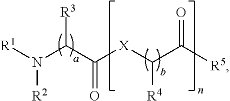

wherein a≥1; b≥1; n≥1; R¹ and R⁵ are each selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —C(═NR^{b})—NR^{c}R^{b}, —OR^{d} and hydroxylalkyl, wherein R^{a}, R^{e} , R^{b} , and R^{d} are each independently selected from hydrogen, —NH₂, alkyl, substituted alkyl, cycloalkyl, substituted cycloalkyl, heterocycloalkyl, substituted heterocycloalkyl, haloalkyl, and acyl; R² is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl; each R³ is selected from amino acid side chains and can be present or absent, or R² and R³ taken together with the nitrogen atom and carbon atom to which they are connected, form a substituted 3- to 8-membered heterocyclic ring; each R⁴ is selected from amino acid side chains and can be present or absent; each X is independently selected from —NR⁶— and —NR⁶—C(═NR⁶)—NR⁶—, and at least one X is —NR⁶—C(═NR⁶)—NR⁶, or R⁴ on the alpha or beta position and the adjacent R⁶ taken together with the nitrogen atom and carbon atom to which they are connected, form a 3- to 8-membered heterocyclic ring; and each R⁶ is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl.

In one embodiment, the subject invention provides a method for synthesizing a peptidomimetic comprising one or more guanidino acids, the method comprising:

1) contacting an amino acid with a guanilidation reagent to form a protected guanidino acid that comprises a guanidine and one or more protection groups, wherein the amino acid is a natural or non-natural, or a modified or non-modified amino acid;

2) removing the one or more protection groups of the protected guanidino acid;

3) mixing the deprotected guanidino acid with a pre-protected amino acid to couple the pre-protected amino acid to the guanidine of the deprotected guanidino acid to form guanidino acids;

4) removing the protection of the product of step 3); and

5) optionally, treating the product of step 4) with the guanilidation regent and repeating steps 2) to 4).

In one embodiment, the subject invention provides a method for synthesizing a peptidomimetic comprising one or more guanidino acids, the method comprising:

1) providing one or more amino acids;

2) contacting each of the one or more amino acids with a guanilidation reagent to form one or more protected guanidino acids, wherein each of the protected guanidine acids comprises one or more protection groups at the guanidine group;

3) removing one or more protection groups of each of the one or more protected guanidino acids; and

4) mixing each of the one or more deprotected guanidine acids in the presence of a coupling reagent.

In some embodiments, the amino acid is selected from natural or non-natural, and modified or non-modified amino acids.

In one embodiment, the guanilidation reagent has a structure of

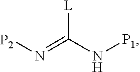

wherein L is a leaving group; P1 and P2 are orthogonal protecting groups, wherein the leaving group is selected from, for example, halogen, —SMe, pyrazole, substituted pyrazole, and ammonium. In specific embodiments, P1 and P2 are each independently selected from, for example, fluorenylmethoxycarbonyl (Fmoc), tert-butyloxycarbonyl (Boc), benzyl chloroformate (Cbz), acetyl (Ac), trifluoroacetyl (TFA), phthalimide, benzyl (Bn), trityl (Trt), benzylideneamine, and tosyl (Ts).

The subject invention further provides materials and methods for intracellularly delivering small molecules such as drugs, nucleic acids, and peptides, as well as proteins and other larger molecules. The subject invention also provides materials and methods for assisting the passage of molecules across biological membranes.

In one embodiment, the peptidomimetics of the subject invention may be conjugated to molecules such as drugs, nucleic acid, peptides, proteins and antibodies, which can be used for treating various diseases.

### BRIEF DESCRIPTION OF DRAWINGS

FIG. 1 shows a scheme of synthesizing peptidomimetics through method A.

FIG. 2 shows a scheme of synthesizing peptidomimetics through method B.

FIG. 3A shows HPLC of the N-TFA, N-Boc guanidino phenyl alanine.

FIG. 3B shows the peak Purity (UV spectra) at different wave lengths.

FIG. 4A shows HPLC of the guanidino dipeptidomimetic obtained from phenylalanine, amino hexanoic acid and phenyl acetic acid.

FIG. 4B shows the mass spectra (LCMS) of the guanidino dipeptidomimetic obtained from Phenylalanine, amino hexanoic acid and phenyl acetic acid.

FIG. 5A shows HPLC of the guanidino dipeptidomimetic obtained from phenylalanine, amino hexanoic acid and acetic anhydride.

FIG. 5B shows the mass spectra (LCMS) of the guanidino dipeptidomimetic obtained from Phenylalanine, amino hexanoic acid and acetic anhydride.

FIG. 6A shows HPLC of the guanidino dipeptidomimetic obtained from valine, amino hexanoic acid and acetic anhydride.

FIG. 6B shows the mass spectra (LCMS) of the guanidino dipeptidomimetic obtained from valine, amino hexanoic acid and acetic anhydride.

FIG. 7A shows HPLC of the guanidino dipeptidomimetic obtained from valine, amino hexanoic acid and phenyl acetic acid.

FIG. 7B shows the mass spectra (LCMS) of the guanidino dipeptidomimetic obtained from valine, amino hexanoic acid and phenyl acetic acid.

FIG. 8A shows HPLC of the guanidino dipeptidomimetic obtained from valine, N-methyl phenylalanine and acetic anhydride.

FIG. 8B shows the mass spectra (LCMS) of the guanidino dipeptidomimetic obtained from valine, N-methyl phenylalanine and acetic anhydride.

FIG. 9A shows HPLC of the guanidino dipeptidomimetic obtained from valine, N-methyl valine and acetic anhydride.

FIG. 9B shows the mass spectra (LCMS) of the guanidino dipeptidomimetic obtained from valine, N-methyl valine and acetic anhydride.

FIG. 10A shows HPLC of the guanidino dipeptidomimetic obtained from leucine, N-methyl valine and acetic anhydride.

FIG. 10B shows the mass spectra (LCMS) of the guanidino dipeptidomimetic obtained from leucine, N-methyl valine and acetic anhydride.

FIG. 11A shows HPLC of the guanidino dipeptidomimetic obtained from leucine, N-methyl alanine and acetic anhydride.

FIG. 11B shows the mass spectra (LCMS) of the guanidino dipeptidomimetic obtained from leucine, N-methyl alanine and acetic anhydride.

FIG. 12A shows HPLC of the guanidino dipeptidomimetic obtained from alanine, N-methyl valine and acetic anhydride.

FIG. 12B shows the mass spectra (LCMS) of the guanidino dipeptidomimetic obtained from alanine, N-methyl valine and acetic anhydride.

FIG. 13A shows HPLC of the guanidino dipeptidomimetic obtained from alanine, N-methyl alanine and acetic anhydride.

FIG. 13B shows the mass spectra (LCMS) of the guanidino dipeptidomimetic obtained from alanine, N-methyl alanine and acetic anhydride.

FIG. 14A shows HPLC of the guanidino dipeptidomimetic obtained from tyrosine, N-methyl valine and acetic anhydride.

FIG. 14B shows the mass spectra (LCMS) of the guanidino dipeptidomimetic obtained from tyrosine, N-methyl valine and acetic anhydride.

FIG. 15A shows HPLC of the guanidino dipeptidomimetic obtained from tyrosine, N-methyl alanine and acetic anhydride.

FIG. 15B shows the mass spectra (LCMS) of the guanidino dipeptidomimetic obtained from tyrosine, N-methyl alanine and acetic anhydride.

FIG. 16A shows HPLC of the guanidino dipeptidomimetic obtained from leucine, beta alanine and acetic anhydride.

FIG. 16B shows the mass spectra (LCMS) of the guanidino dipeptidomimetic obtained from leucine, beta alanine and acetic anhydride.

FIG. 17A shows HPLC of the guanidino dipeptidomimetic obtained from valine, leucine and acetic anhydride.

FIG. 17B shows the mass spectra (LCMS) of the guanidino dipeptidomimetic obtained from valine, leucine and acetic anhydride.

FIG. 18A shows the structure of the acetylated tripeptidomimetic 2-CTC-104.

FIG. 18B shows HPLC of the acetylated tripeptidomimetic obtained from Guanidino leucine, tyrosine, proline and acetic anhydride.

FIG. 18C shows the Tic-Spectrum of acetylated tripeptidomimetic obtained from Guanidino leucine, tyrosine, proline and acetic anhydride.

FIG. 18D show the mass spectra (LCMS) of the acetylated tripeptidomimetic obtained from Guanidino leucine, tyrosine, proline and acetic anhydride.

FIG. 19A shows the structure of the acetylated tripeptidomimetic 2-CTC-128.

FIG. 19B shows HPLC of the acetylated tripeptidomimetic obtained from leucine, guanidino tyrosine, proline and acetic anhydride.

FIG. 19C shows the TIC spectra of the acetylated tripeptidomimetic obtained from leucine, guanidino tyrosine, proline and acetic anhydride.

FIG. 19D show the MS spectra of the acetylated tripeptidomimetic obtained from leucine, guanidino tyrosine, proline and acetic anhydride.

### DETAILED DISCLOSURE

The subject invention provides compounds, peptidomimetics, and methods of synthesis thereof. The subject invention provides an unexplored field in peptidomimetic chemistry, the synthesis and use of guanidino acids and/or poly guanidino acids in drug discovery, not only as vehicles for drug delivery but as toolbox for drug discovery.

In one embodiment, the subject invention provides a modified amino acid comprising a guanidine group. The amino acids can be natural or non-natural, modified or non-modified amino acids or analogs thereof. The amino acids may be, for example, α-, β-, γ- or Δ-amino acids. In specific embodiments, the amino acids are selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, selenocysteine, pyrrolysine, and derivatives and analogues thereof. Other examples of amino acid derivatives or analogues include, for example, amino hexanoix acid, aminocaproic acid, aminovaleric acid, aminooctanoic acid, N-methyl phenylalanine, N-methyl valine, and N-methyl alanine.

In one embodiment, the modified amino acid is a guanidino acid. The guanidino acid can be an α-, β-, γ- or Δ- guanidino acid. For example, the α-guanidino acid has a general structure of

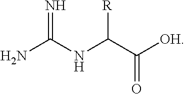

The β-guanidino acid has a general structure of

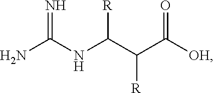

wherein each R represents any group or amino acid side chain.

In one embodiment, the subject invention provides a peptide or peptidomimetic comprising one or more guanidino acids. The alteration of conventional peptides to peptidomimetics encompasses side-chain manipulation, turn-mimics, amino acid extension, and backbone modifications. Particularly attractive, are peptide bond surrogates in which peptide bonds (amides) have been replaced with other chemical groups.

In one embodiment, the subject invention provides a peptidomimetic comprising oligo(amino acid)s or poly(amino acid)s, wherein the amino acid residues in the oligo(amino acid)s or poly(amino acid)s are connected with guanidines as peptide bond surrogates.

The incorporation of the guanidine as amide bond surrogates offers significant differences in polarity, hydrogen bonding capability, and acid-base character. The introduction of such modifications to the peptide backbone provides a new class of peptidomimetics that address peptide and existing peptidomimetics limitations by displaying improved bioavailability, metabolic stability, duration of action, enhanced receptor affinity and selectivity and, especially, improved cell penetrating potential. This novel class of compounds opens a vast range of opportunities associated with intracellular and tissue targets protected by other barriers (e.g., BBB, ocular, lung, skin, aural).

In one embodiment, the peptidomimetic according to the subject invention comprises a chain of oligo(amino acid)s or poly(amino acid)s having one or more guanidine groups incorporated in the backbone of oligo(amino acid)s or poly(amino acid)s. The guanidine surrogate can be inserted at any position of the backbone of oligo(amino acid)s or poly(amino acid)s.

In some embodiments, the peptidomimetic has a structure from N-terminal to C-terminal as [(Xaa)m-Guanidine-(Xaa)o]p, wherein Xaa is any amino acid; m≥0, o≥0, and p≥1; wherein m and o are not 0 at the same time; and wherein the N-terminal can be, for example, a free amine, modified amine such as alkylated amine, and acylated amine, or guanidine; the C-terminal can be, for example, a free carboxylic acid, ester, amind, thioester, or carbonyl guanidine.

In some embodiments, the N-terminal of the peptidomimetic can be, for example, a free amine, modified amine such as alkylated amine, and acylated amine, or guanidine; and the C-terminal of the peptidomimetic can be, for example, a free carboxylic acid, or ester.

In one embodiment, the peptidomimetic has a structure of

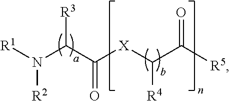

wherein a≥1; b≥1; n≥1; R¹ and R⁵ are each selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a},—NR^{c}R^{b}, —C(═NR^{b})—NR^{c}R^{b}, —OR^{d} and hydroxylalkyl, wherein R^{a}, R^{c} , R^{b} , and R^{d} are each independently selected from hydrogen, —NH₂, alkyl, substituted alkyl, aryl, substituted aryl, heteroalkyl, substituted heteroalkyl, cycloalkyl, substituted cycloalkyl, heterocycloalkyl, substituted heterocycloalkyl, haloalkyl, and acyl; R² is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl; each R³ is selected from any functional groups, hydrogen and amino acid side chains, and can be present or absent, or R² and R³ taken together with the nitrogen atom and carbon atom to which they are connected, form a substituted or unsubstituted 3- to 8-membered heterocyclic ring; each R⁴ is selected from any functional groups, hydrogen and amino acid side chains, and can be present or absent; each X is independently selected from —NR⁶— and —NR⁶—C(═NR⁶)—NR⁶—, and at least one X is —NR⁶—C(═NR⁶)—NR⁶, or R⁴ on the alpha or beta position and the adjacent R⁶ taken together with the nitrogen atom and carbon atom to which they are connected, form a substituted or unsubstituted 3- to 8-membered heterocyclic ring; and each R⁶ is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl.

In a preferred embodiment, a is 1-10, each b is 1-10, and n is 1-20.

In specific embodiments, R² and R³ taken together with the nitrogen atom and carbon atom to which they are connected, form a substituted or unsubstituted 3-, 4-, 5-, 6- 7-, or 8-membered heterocyclic ring, preferably, 5- or 6-membered heterocyclic ring; and R⁴ on the alpha or beta position and the adjacent R⁶ taken together with the nitrogen atom and carbon atom to which they are connected, form a substituted or unsubstituted 3-, 4-, 5-, 6- 7-, or 8-membered heterocyclic ring, preferably, 5- or 6-membered heterocyclic ring.

In some embodiments, the N-terminal of the peptidomimetic can be, for example, a free amine, modified amine such as alkylated amine, and acylated amine, or guanidine; and the C-terminal of the peptidomimetic can be, for example, a free carboxylic acid, ester, amide, thioester, or carbonyl guanidine.

In certain embodiments, the N- and/or C-terminal of the peptidominetic may comprise mono, bi, or trifunctional linker groups comprising, for example, hydrazide, hydrazone, esters (e.g., NHS-ester), amides, azides, oxyme, meleimide, b-iodo, or —SH.

In a specific embodiment, R¹ and R² are each independently selected from hydrogen, alkyl, acyl, and —C(NH)NH₂. R⁵ is —SR^{a}, —NR^{c}R^{b}, or —OR^{d} , wherein R^{a}, R^{c} , R^{b} , and R^{d} are each independently selected from hydrogen, —NH₂, alkyl, substituted alkyl, aryl, substituted aryl, heteroalkyl, substituted heteroalkyl, cycloalkyl, substituted cycloalkyl, heterocycloalkyl, substituted heterocycloalkyl, haloalkyl, and acyl.

In one embodiment, the oligo(amino acid)s or poly(amino acid)s may be a peptide/protein that is functional or nonfunctional, and/or therapeutic or non-therapeutic. The amino acid may be natural or non-natural, modified or non-modified amino acids or analogs thereof. The amino acid may be, for example, an α-, β-, γ-, or Δ-amino acid.

The oligo(amino acid)s or poly(amino acid)s can be peptides with any length. The number of amino acid in the oligo(amino acid)s or poly(amino acid)s may be, for example, at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or any number therebetween.

In one embodiment, the peptidomimetic has a structure of

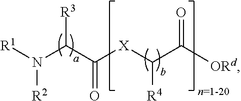

wherein a≥1; b≥1; R¹ is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —NR^{c}R^{b}, —C(═NR^{b})—NR^{c}R^{b}, —OR^{d} and hydroxylalkyl, wherein R^{a}, R^{c} , R^{b}, and R^{d} are each independently selected from hydrogen, —NH₂, alkyl, substituted alkyl, aryl, substituted aryl, heteroalkyl, substituted heteroalkyl, cycloalkyl, substituted cycloalkyl, heterocycloalkyl, substituted heterocycloalkyl, haloalkyl, and acyl; R² is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, SR^{a}, —NR^{c}R^{b}, —OR^{d} and hydroxylalkyl; each R³ is selected from any functional groups, hydrogen and amino acid side chains, and can be present or absent, or R² and R³ taken together with the nitrogen atom and carbon atom to which they are connected, form a substituted or unsubstituted 5- or 6-membered heterocyclic ring; each R⁴ is selected from any functional groups, hydrogen and amino acid side chains, and can be present or absent; each X is independently selected from —NR⁶—and —NR⁶—C(═NR⁶)—NR⁶—, and at least one X is -NR⁶—C(═NR⁶)—NR⁶, or R⁴ on the alpha or beta position and the adjacent R⁶ taken together with the nitrogen atom and carbon atom to which they are connected, form a substituted or unsubstituted 3- to 8-membered heterocyclic ring; and each R⁶ is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl.

In a preferred embodiment, a is 1-10, each b is 1-10, and each X is independently selected from —NH—and —NH—C(═NH)—NH—, and at least one X is —NH—C(═NH)—NH—.

In some embodiments, the peptidomimetic has a structure of

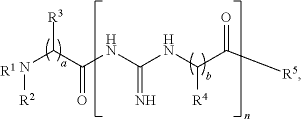

wherein a≥1; b≥1; n≥1; R¹ and R⁵ are each selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —C(═NR^{b})—NR^{c}R^{b}, —OR^{d} and hydroxylalkyl, wherein R^{a}, R^{c} , R^{b} , and R^{d} are each independently selected from hydrogen, —NH₂, alkyl, substituted alkyl, aryl, substituted aryl, heteroalkyl, substituted heteroalkyl, cycloalkyl, substituted cycloalkyl, heterocycloalkyl, substituted heterocycloalkyl, haloalkyl, and acyl; R² is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl; each R³ is selected from any functional groups, hydrogen and amino acid side chains, and can be present or absent, or R² and R³ taken together with the nitrogen atom and carbon atom to which they are connected, from a 5- or 6-membered heterocyclic ring; and each R⁴ is selected from any functional groups, hydrogen and amino acid side chains, and can be present or absent.

In some embodiments, the peptidomimetic has a structure of

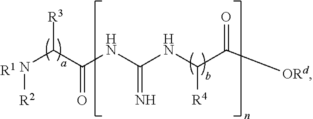

wherein a≥1; b≥1; n≥1; R¹ is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —C(═NR^{b})—NR^{c}R^{b}, —OR^{d} and hydroxylalkyl, wherein R^{a}, R^{c}, R^{b}, and R^{d} are each independently selected from hydrogen, —NH₂, alkyl, substituted alkyl, aryl, substituted aryl, heteroalkyl, substituted heteroalkyl, cycloalkyl, substituted cycloalkyl, heterocycloalkyl, substituted heterocycloalkyl, haloalkyl, and acyl; R² is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl; each R³ are selected from any functional groups, hydrogen and amino acid side chains, and can be present or absent, or R² and R³ taken together with the nitrogen atom and carbon atom to which they are connected, form a 5- or 6-membered heterocyclic ring; and each R⁴ is selected from any functional groups, hydrogen and amino acid side chains, and can be present or absent.

In one embodiment, the peptidomimetic has a structure of

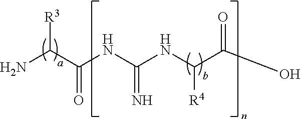

wherein a≥1, preferably, a is 1-10; b≥1, preferably, b is 1-10; n≥1, preferably, n is 1-20; each R³ are selected from amino acid side chains, and can be present or absent; and each R⁴ is selected from amino acid side chains, and can be present or absent.

In one embodiment, the peptidomimetic has a structure of:

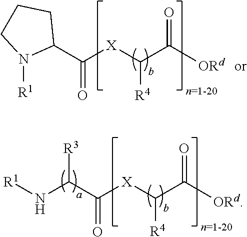

In one embodiment, the peptidomimetic is a guanidino dipeptide, guanidino tripeptide, guanidino tetrapeptide, guanidino pentapeptide or guanidino hexapeptide.

In specific embodiments, the peptidomimetic is a guanidino dipeptide, for example,

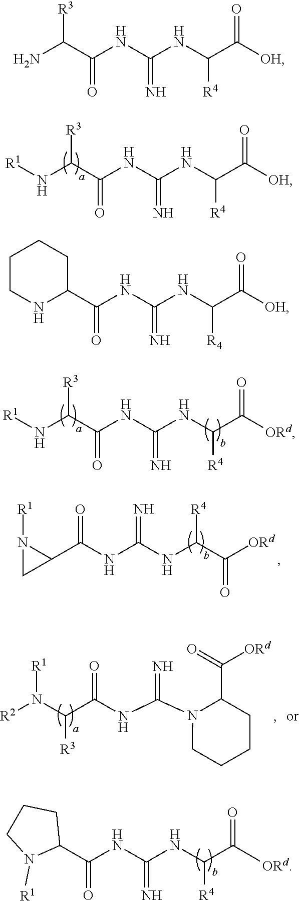

In specific embodiments, the peptidomimetic has a structure of, for example,

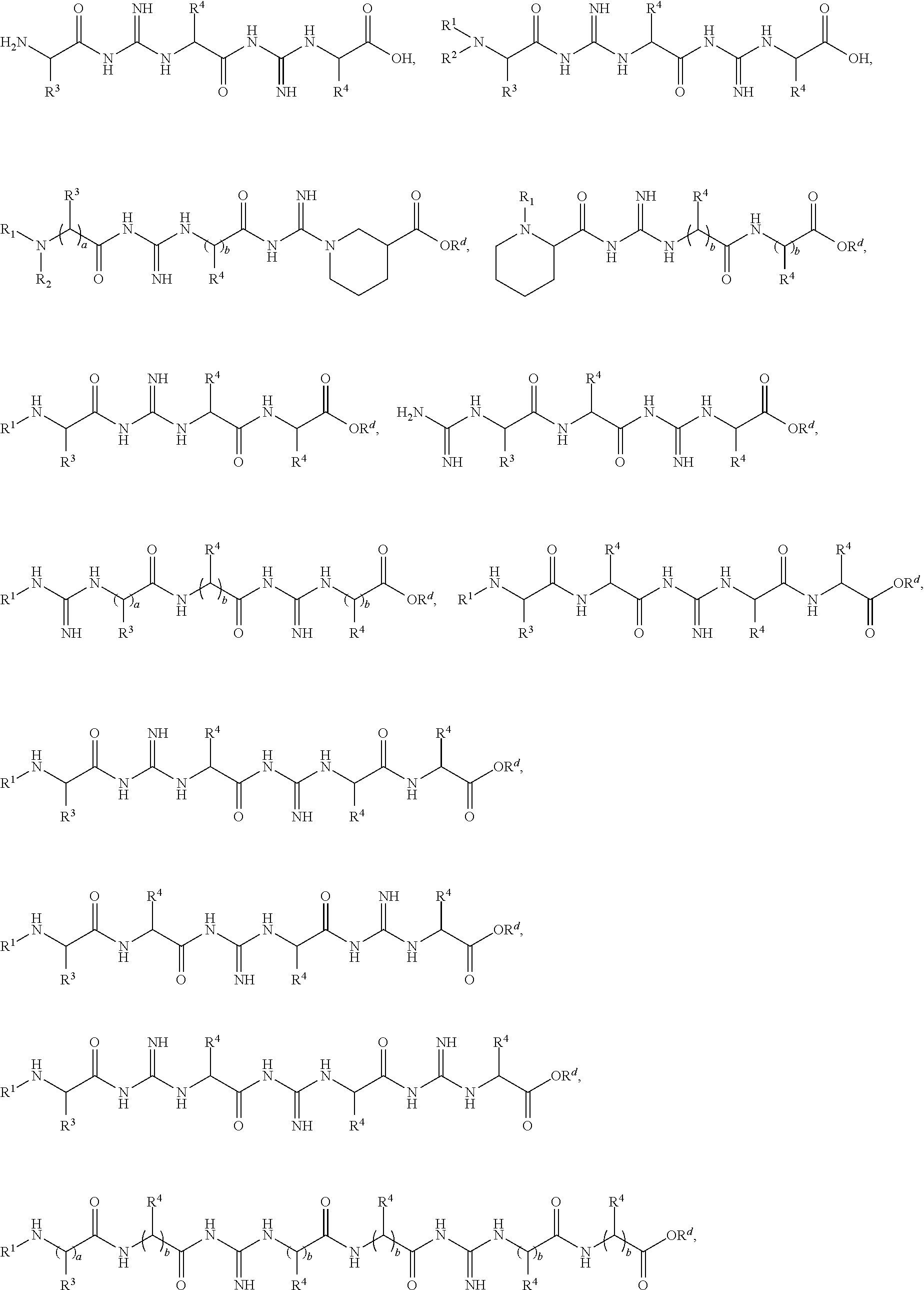

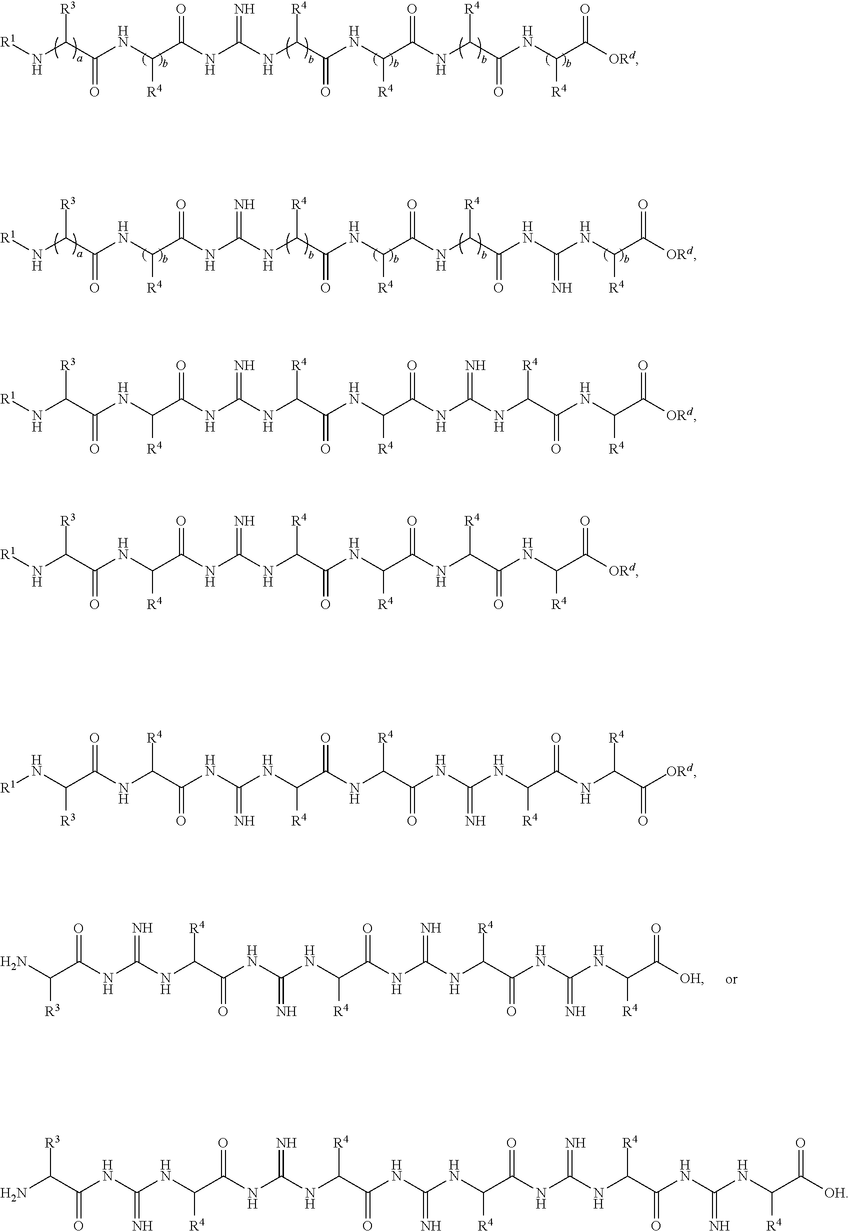

In specific embodiments, the peptidomimetic is a guanidino dipeptide or tripeptide selected from, for example,

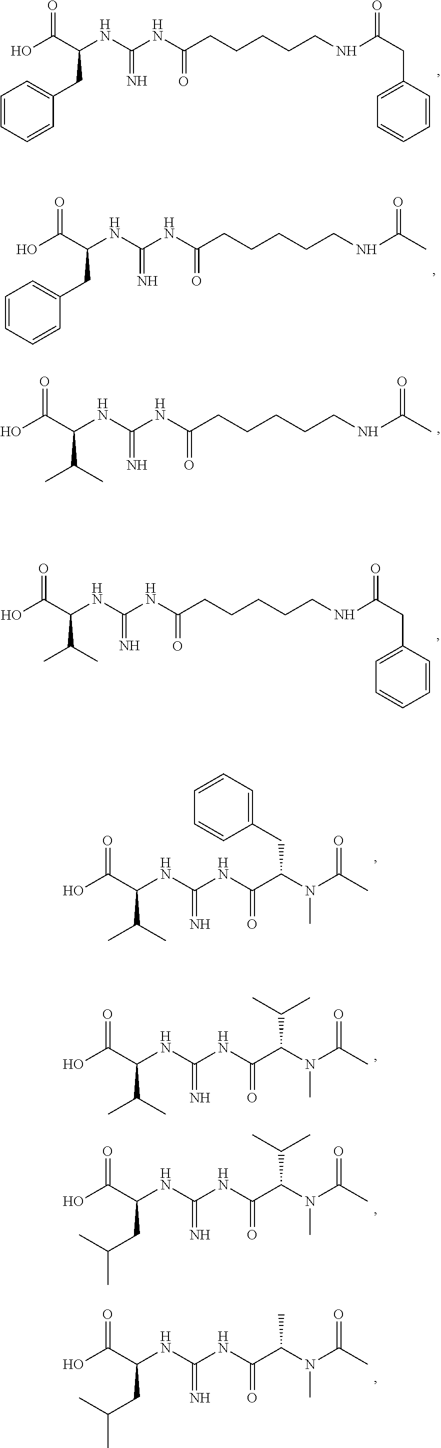

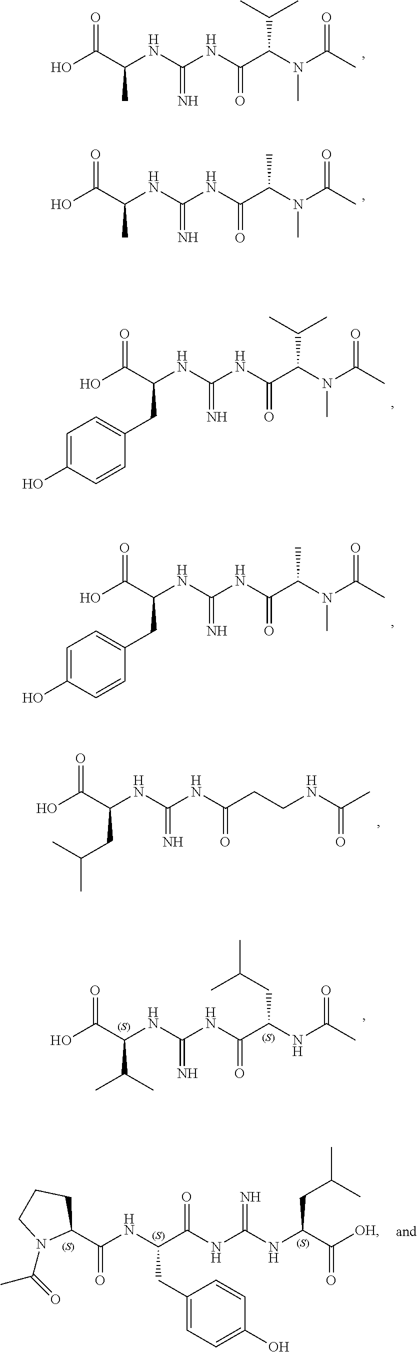

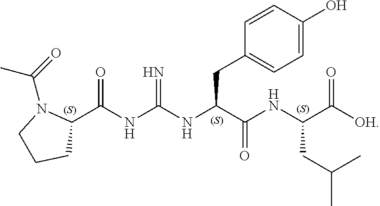

In one embodiment, the subject invention provides methods for synthesizing the peptidomimetics described herein. The methods take advantage of the amino acid functionality and stereochemistry that is commercially available as standard Fmoc/Boc amino acids, as well as the benefits of solid phase peptide synthesis.

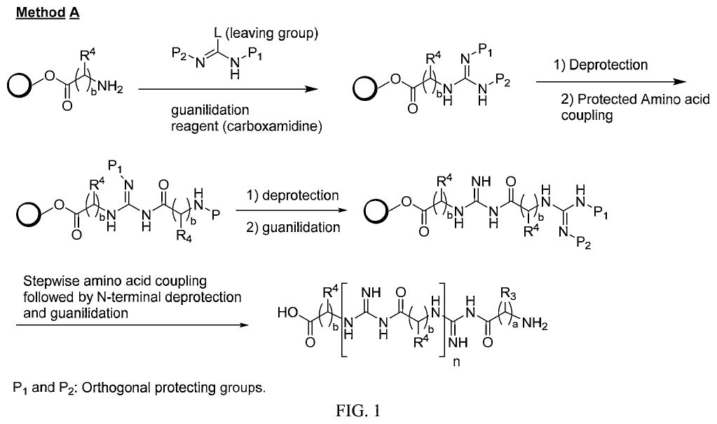

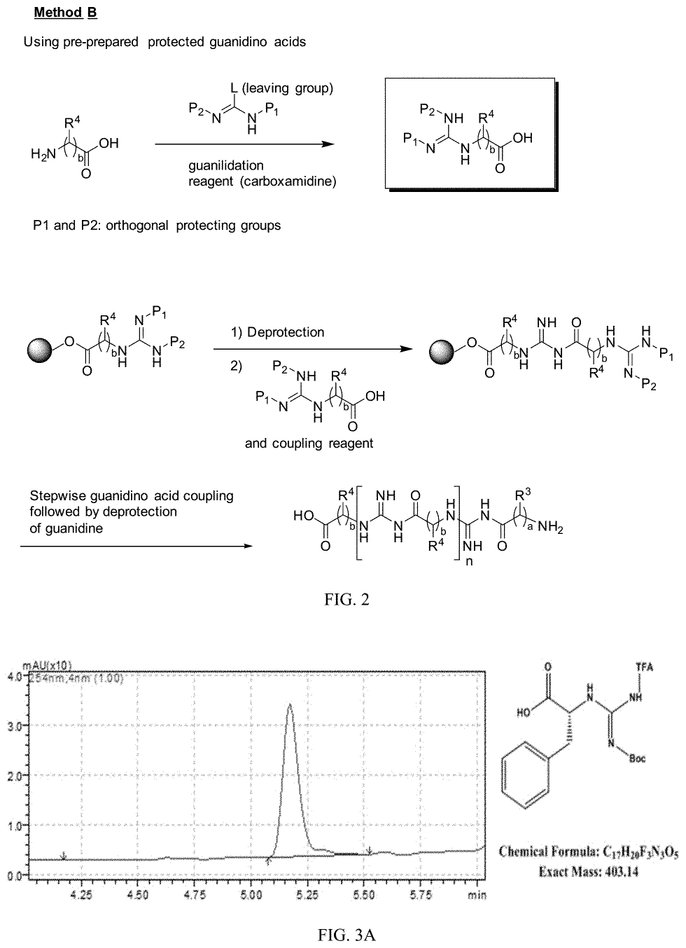

In one embodiment, the synthesis method involves stepwise coupling of protected amino acid(s), e.g., α-amino acid(s) to N-terminal pre-generated guanidine (method A, FIG. 1), or stepwise coupling of pre-prepared orthogonally protected guanidino acid(s), e.g., α-guanidino acid(s) (method B, FIG. 2) such that the pre-prepared orthogonally protected guanidino acid(s) can be used as building blocks for the direct insertion of guanidino acid.

In specific embodiments, the protected guanidino acid has a structure of

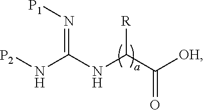

wherein a≥1; R is selected from any functional group and amino acid side chains; P1 and P2 are each a protecting group.

In one embodiment, the subject invention provides a method for synthesizing a peptidomimetic of the subject invention, the method comprising a plurality of cycles of steps including:

1) forming protected guanidino acid(s) comprising a N-terminal guanidine group with one or more protection groups;

2) deprotecting the guanidine acid(s) by removing the protection at the N-terminal guanidine group and coupling a pre-protected amino acid, or a pre-protected guanidino acid to the deprotected N-terminal guanidine group; and 3) removing the protection including pre-protection, and the C- and/or N-terminal protection.

In one embodiment, the step of forming protected guanidino acid(s) comprises contacting an amino acid, or guanidino acid(s) comprising a free —NH₂ group, with a guanilidation reagent, wherein the amino acid or guanidino acid(s) can be pre-protected at —COOH and/or —NH₂ end, or unprotected.

In some embodiments, the protected amino acid or guanidine acid(s), without the treatment of the guanilidation reagent, comprises a free-NH₂ group at the N-terminal, which can form a peptide bond with the pre-protected amino acid during the coupling step.

In one embodiment, the subject invention provides a method for synthesizing a guanidino dipeptide, the method comprising:

1) contacting an amino acid with a guanilidation reagent to form a protected guanidino acid that comprises a guanidine with one or more protection groups;

2) removing one or more of protection groups of the guanidine of the guanidino acid;

3) coupling a pre-protected amino acid to the deprotected guanidine of the guanidino acid; and

4) removing the protection of the product of step 3) to form a guanidino dipeptide.

In one embodiment, the subject invention provides a method for synthesizing a guanidino tripeptide, the method comprising:

1) contacting an amino acid with a first guanilidation reagent to form a protected guanidino acid that comprises a guanidine with one or more protection groups;

2) removing one or more of protection groups of the guanidine of the guanidino acid;

3) coupling a pre-protected amino acid to the deprotected guanidine of the guanidino acid;

4) removing the pre-protection of the product of step 3) to form a guanidino dipeptide;

5) contacting the guanidine dipeptide with a second guanilidation reagent to form a protected guanidino acids that comprises a N-terminal guanidine with one or more protection groups;

6) removing one or more of protection groups of the N-terminal guanidine of the protected guanidino acids;

7) mixing the product of step 6) with a second pre-protected amino acid to couple the second pre-protected amino acid to the deprotected N-terminal guanidine of the guanidino acids; and

8) removing the protection of the product of step 7) to form a guanidino tripeptide.

In specific embodiments, the protection group is selected from for example, fluorenylmethoxycarbonyl (Fmoc), tert-butyloxycarbonyl (Boc), benzyl chloroformate (Cbz), acetyl (Ac), trifluoroacetyl (TFA), phthalimide, benzyl (Bn), trityl (Trt), benzylideneamine, and tosyl (Ts).

In one embodiment, the starting amino acid and the pre-protected amino acid (e.g., the first pre-protected amino acid, the second pre-protected amino acid) can be the same or different. In one embodiment, the first and second guanilidation reagents can be the same or different.

In one embodiment, the subject invention provides a method for synthesizing a polypeptide or peptidomimetic comprising one or more guanidino acids, the method comprising:

1) contacting an amino acid with a guanilidation reagent to form a protected guanidino acid that comprises a guanidine with one or more protection groups;

2) removing one or more of protection groups of the guanidine of the protected guanidino acid;

3) adding a pre-protected amino acid to couple the pre-protected amino acid to the N-terminal of the deprotected guanidino acid, e.g., N-terminal guanidine or N-terminal —NH₂ group to form guanidino acids;

4) removing the protection, including pre-protection and the C- and/or N-terminal protection, of the product of step 3);

5) optionally, treating the product of step 4) with the guanilidation reagent, and

6) repeating steps 2) to 4), or 3) to 4).

In one embodiment, the starting amino acid and the pre-protected amino acid coupled to the guanidine acid(s) in each cycle can be the same or different.

In one embodiment, the subject invention also provides a method for synthesizing a polypeptide or peptidomimetic comprising one or more guanidino acids, the method comprising:

1) providing one or more amino acids,

2) contacting each of the one or more amino acids with a guanilidation reagent to form one or more protected guanidino acids, wherein each of the protected guanidine acids comprises one or more protection groups at the guanidine group;

3) removing one or more protection groups of each of the one or more protected guanidino acids; and

4) mixing each of the one or more deprotected guanidine acids in a stepwise manner in the presence of a coupling reagent, wherein the coupling reagent can be any coupling reagent in peptide synthesis known in the art, for example, N,N-disubstituted carbodiimides, acyl chloride, acyl fluoride, anhydrides, BOP, PyBOP, PyAOP, PyOxim, TOTU, TSTU, COMU, DEPBT, HBTU, HATU, PyAOP, and HCTU.

In one embodiment, the subject invention also provides a method for synthesizing a polypeptide or peptidomimetic comprising one or more guanidino acids, the method comprising:

preparing one or more pre-protected guanidino acids comprising one or more protection groups at the guanidine group;

coupling the one or more pre-protected guanidino acids in a stepwise manner in the presence of a coupling reagent; and

removing the one or more protection groups.

In one embodiment, the step of preparing the pre-protected guanidino acid comprises contacting an amino acid with a guanilidation reagent.

In one embodiment, the step of coupling the pre-protected guanidino acids in the stepwise manner comprises repetitive steps of removing the one or more protection groups of a first pre-protected guanidino acid(s); and mixing the first deprotected guanidino acid(s) with a second pre-protected guanidino acid in the presence of a coupling reagent.

In one embodiment, the guanilidation reagent is a carboxaminine having a structure of

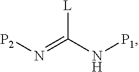

wherein L is a leaving group; P1 and P2 are orthogonal protecting groups. In specific embodiments, the leaving group is selected from, for example, halogen, —SMe, pyrazole, substituted pyrazole, and ammonium. In specific embodiments, P1 and P2 are each independently selected from, for example, fluorenylmethoxycarbonyl (Fmoc), tert-butyloxycarbonyl (Boc), benzyl chloroformate (Cbz), acetyl (Ac), trifluoroacetyl (TFA), phthalimide, benzyl (Bn), trityl (Trt), benzylideneamine, and tosyl (Ts).

In specific embodiments, the guanilidation reagent can be selected from N-Boc-N′-TFA-pyrazole-1-carboxamidine

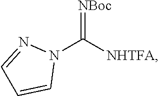

1,3-Bis(tert-butoxycarbonyl)-2-methyl-2-thiopseudourea

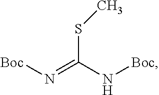 [SMILES: CS/C(=N\C(=O)OC(C)(C)C)NC(=O)OC(C)(C)C]

and 1,3-Bis(tert-butoxycarbonyl)guanidine

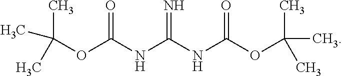 [SMILES: CC(C)(C)OC(=O)NC(=N)NC(=O)OC(C)(C)C]

In one embodiment, the method of the subject invention further comprises a step of modifying the N-terminal and/or C-terminal of the peptidomimetic so that the N-terminal of the peptidomimetic can be, for example, a modified amine such as alkylated amine, and acylated amine, or guanidine, and the C-terminal of the peptidomimetic can be, for example, an ester, amide, thioester, or carbonyl guanidine.

In certain embodiments, each step of the methods of the subject invention occurs in a solvent or a mixture of solvents. Such solvent may be selected from, for example, DMF, DIC, DCM, alcohol such as MeOH, water and a combination thereof.

The subject invention also provides methods for intracellularly delivering compounds, or peptidomimetics, as drugs, or drug carriers, across biological membranes

In one embodiment, the subject invention provides a therapeutic formulation comprising the peptidomimetic of the subject invention and a pharmaceutically acceptable carrier, and optionally, the therapeutic formulation further comprising one or more active agents.

“Pharmaceutically acceptable carrier” refers to a diluent, adjuvant or excipient with which the one or more active agents disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith.

Examples of carriers suitable for use in the pharmaceutical compositions are known in the art and such embodiments are within the purview of the invention. The pharmaceutically acceptable carriers and excipients, including, but not limited to, aqueous vehicles, water-miscible vehicles, non-aqueous vehicles, stabilizers, solubility enhancers, isotonic agents, buffering agents, suspending and dispersing agents, wetting or emulsifying agents, complexing agents, sequestering or chelating agents, cryoprotectants, lyoprotectants, thickening agents, pH adjusting agents, and inert gases. Other suitable excipients or carriers include, but are not limited to, dextran, glucose, maltose, sorbitol, xylitol, fructose, sucrose, and trehalose.

The compositions can be administered to a subject by methods including, but not limited to, (i) administration through oral pathways, which administration includes administration in capsule, tablet, granule, spray, syrup, or other such forms; (ii) administration through non-oral pathways, which administration includes administration as an aqueous suspension, an oily preparation or the like or as a drip, suppository, salve, ointment or the like; administration via injection, subcutaneously, intraperitoneally, intravenously, intramuscularly, intradermally, or the like; as well as (iii) administration topically, or as deemed appropriate by those of skill in the art for bringing the compound into contact with living tissue; and (iv) administration via controlled released formulations, depot formulations, and infusion pump delivery.

The term “subject” or “patient,” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof), such as “comprising,” “comprises,” and “comprise,” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of ” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

As used herein, each of a, b, m, n, o, and p is intended to include ≥0, ≥1, ≥2, ≥3, ≥4, ≥5, ≥6, ≥7, ≥8, ≥9, ≥10, ≥11, ≥12, ≥13, ≥14, ≥15, ≥16, ≥17, ≥18, ≥19, ≥20, ≥21, ≥22, ≥23, ≥24, ≥25, ≥26, ≥27,≥28, ≥29, ≥30, ≥31,≥32, ≥33, ≥34, ≥35, ≥36, ≥37, ≥38, ≥39, ≥40, ≥41, ≥42, ≥43, ≥44, ≥45,≥46, ≥47, ≥48, ≥49, ≥50, ≥51, ≥52, ≥53, ≥54, ≥55, ≥56, ≥57, ≥58, ≥59, ≥60, ≥61, ≥62, ≥63, ≥64, ≥65, ≥66, ≥67, ≥68, ≥69, ≥70, ≥71, ≥72, ≥73, ≥74, ≥75, ≥76, ≥77, ≥78, ≥79, ≥80, ≥81, ≥82, ≥83, ≥84, ≥85, ≥86, ≥87, ≥88, ≥89, ≥90, ≥91, ≥92, ≥93, ≥94, ≥95, ≥96, ≥97, ≥98, ≥99, ≥100 and any value therebetween.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### EXAMPLES

### Chemicals

N-Boc-N′-TFA-pyrazole-1-carboxamidine was purchased from Sigma Aldrich (St. Louis, Mo.), Fmoc amino acids, cand all other reagents were purchased from Chem Impex (Wood Dale Ill.). The solvents were purchased from Fisher Scientific (Pittsburgh, Pa.). The LC/MS Spectra were recorded in Shimadzu 2010 LC/MS system, software version 3. Mobile phases used were either HPLC grade or LC/MS grade purchased from Sigma Aldrich and Fisher Scientific.

### Synthesis of Guanidine Dipeptides

2-Chlorotrityl resin (100mg) bags were prepared and swelled in DCM (30 minutes), to which Fmoc (Phenylalanine, Leucine, Valine, Alanine and Tyrosine) amino acids (4 eq.), N, N-Diisopropylethylamine (DIEA, 6 eq.) were added to the anhydrous DCM. The reaction mixture along with respective bags was stirred at room temperature for up to 3 hours ⁽¹⁾. The reaction mixture was dumped and washed with DCM, DMF and DCM (2×). Further, the bags were treated with 17:2:1 (DCM: MeOH: DIPEA) for another 30 minutes as scavenger to avoid side products ⁽²⁾. The bags were then washed with MeOH: DCM (1:1, 3×) and DCM (3×). The Fmoc-amino acids were treated with 20% Piperidine/DMF (2×, 10 minutes) for Fmoc removal and guanidine coupling with N-Boc-N′-TFA-pyrazole-1-carboxamidine (2 eq.) overnight in anhydrous DMF. After completion of the reaction, the washing was made with DMF and DCM (2×).

The amino acid coupled bags with guanidine were protected with Boc and TFA, where TFA was deprotected using Cesium carbonate (CsCO₃) or Potassium carbonate (K₂CO₃) for 3 hours in MeOH: H₂O (5:2). Since the resin needs to be swollen, small quantity of DMF was used. The washing was made with MeOH: H₂(3×), DMF and DCM (2×). After TFA deprotection, the bags were treated with various Fmoc amino acids (normal, beta and methylated amino acids, 5 eq.), N, N-Dicyclohexylcarbodiimide (DIC, 5 eq.) and anhydrous DMF at 70° C. for 4 hrs. The washing of bags was finished with DMF and DCM (2×). The Fmoc was removed with 20% Piperidne/DMF (1×, 10 minutes) and straightaway those bags were treated with Acetic anhydride, DIEA (50 eq.) for 1 hr. in anhydrous DMF to prevent further interaction between guanidine and amino group. After completion of the reaction, the bags were washed with DMF and DCM (2×). Finally, 5% TFA/DCM was used to cleave products from resin (5 mL, 2×). The solution was collected and concentrated TFA was added to cleave Boc from the product. The solution was evaporated in Rota vapor and lyophilized.

### LC/MS Analysis

The peptide reaction progress and its purity were monitored in Shimadzu 2010 LC/MS system, software version 3, with a DGU-20A degasser unit, LC-20AD binary solvent pumps, a SIL-20A HT auto sampler and a CTO-20A column oven. A Shimadzu SPD-M20A diode array detector was used for detections. 214 nm and 254 nm spectral wavelengths were customized for UV/PDA chromatogram. A Phenomenex Luna C18 analytical column (5 μm, 50×4.6 mm i.d.) was used for the chromatographic separations/purity of peptides/compounds. The column was guarded with a Phenomenex C18 column guard (5 μm, 4×3.0 mm i.d.). The LC/MS grade Acetonitrile/water (both with 0.1% formic acid) solvents were used as the mobile phases for chromatographic separation/detection. The analysis method was assigned with initial 5% Acetonitrile (v/v), that got increased linearly to 55% Acetonitrile over 12 minutes. Further, the mobile phase was gradient to 95% Acetonitrile and remained for 2 minutes which was then being linearly decreased to 5%. The flow rate was kept 0.5 mL/minute throughout the run. The temperature for column oven and flow cell for the diode array detector was 30° C. The temperature for the auto sampler temperature was 15° C. The samples were diluted in 50% (v/v) of ACN: H₂O and 5 uL-10 uL of sample was injected for analysis.

### Example 1—Synthesis of Guanidine Monopeptide in Solution

L-Phenylalanine amino acid (0.6053 mmol, 100 mg) and N-Boc-N′-TFA-pyrazole-1-carboxamidine (0.6354 mmol, 194.6 mg) were dissolved in anhydrous DMF (1 ml). Becausee the mixture did not dissolve absolutely, CsCO₃ (0.6052 mmol, 197.2 mg) and H₂O were added (1 mL). The reaction mixture was stirred overnight at room temperature. The reaction completion was checked in LCMS. The solution was diluted with 50:50 (ACN and water) and lyophilized.

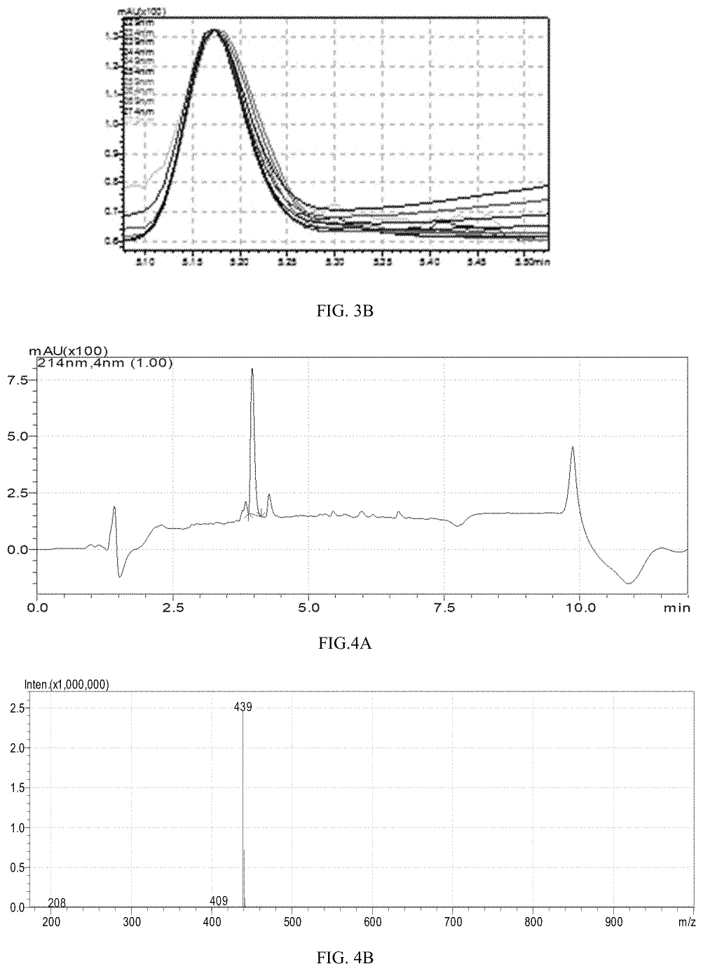

The deprotection of N-TFA, N-Boc guanidino phenyl alanine (FIG. 3A) affords guanidino phenyl alanine. FIG. 3B shows the peak purity of N-TFA, N-Boc guanidino phenyl alanine at different wavelengths.

### Example 2—Synthesis of Acylated Dipeptidomimetics

The guanidino dipeptidomimetic, Phenylalanine-gua-amino hexanoic-phenyl acetic acid (FIGS. 4A and 4B), is obtained from Phenylalanine, amino hexanoic acid and phenyl acetic acid as shown in Scheme 1 below:

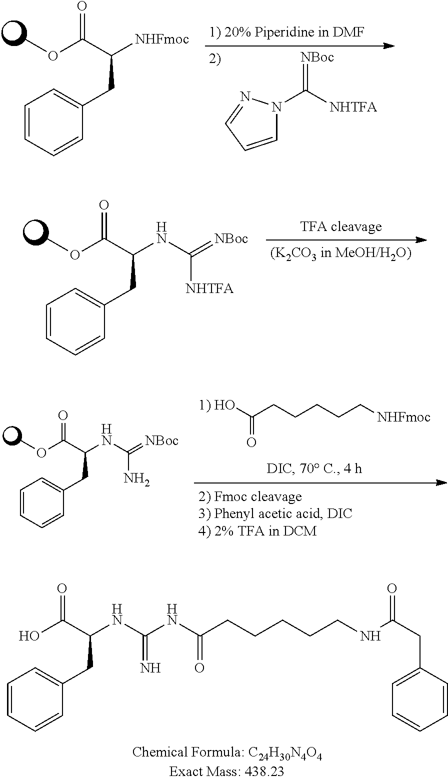

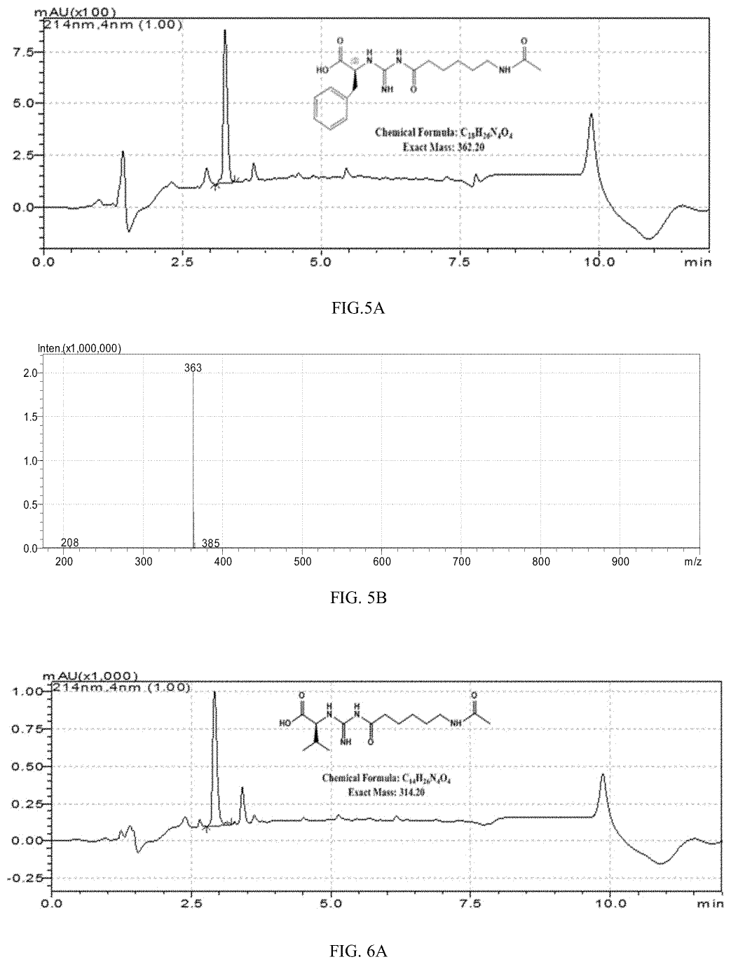

Phenylalanine-Gua-amino hexanoic-Acetic anhydride (FIGS. 5A and 5B) can be obtained from phenylalanine, amino hexanoic acid and acetic anhydride as shown in Scheme 2 below:

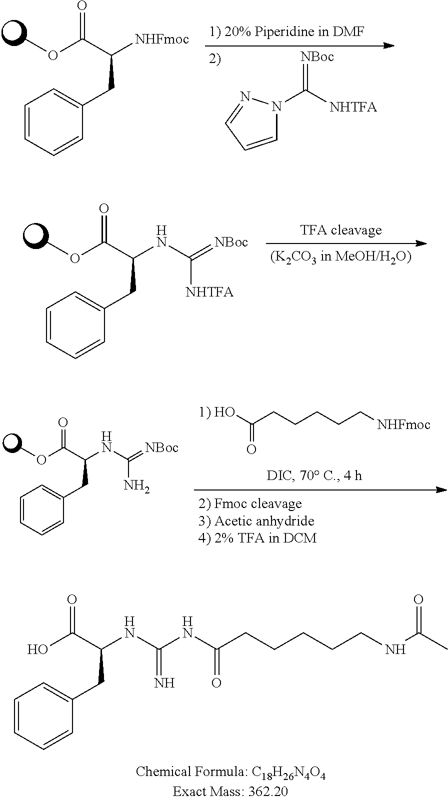

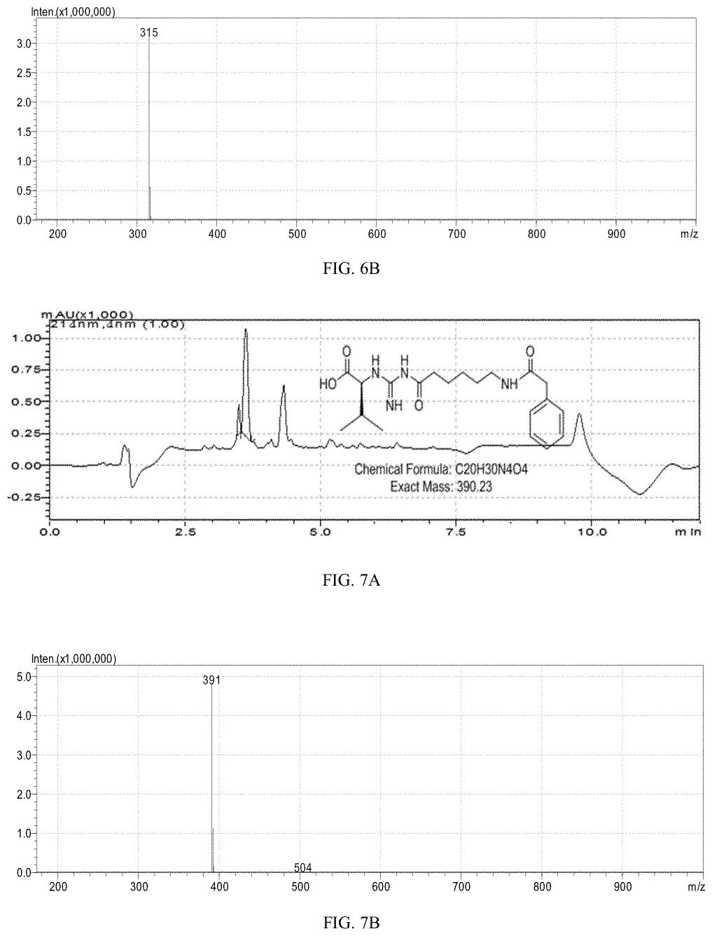

Valine-Gua-amino hexanoic-Acetic Anhydride (FIGS. 6A and 6B) can be obtained from valine, amino hexanoic acid and acetic anhydride as shown in Scheme 3 below:

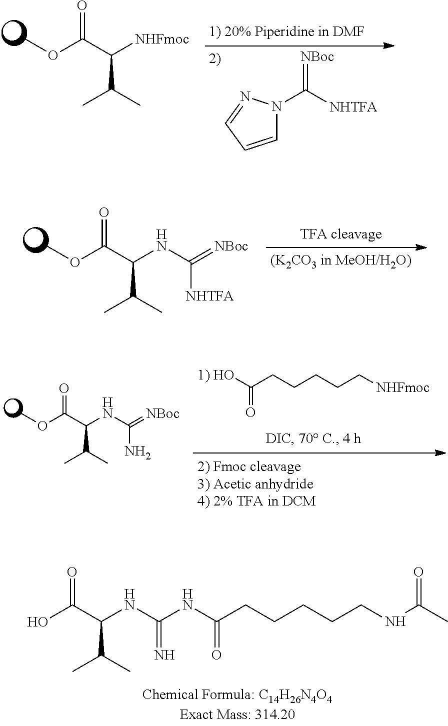

Valine-Gua-amino hexanoic-Phenyl Acetic Anhydride (FIGS. 7A and 7B) can be obtained from valine, amino hexanoic acid and phenyl acetic acid as shown in Scheme 4 below:

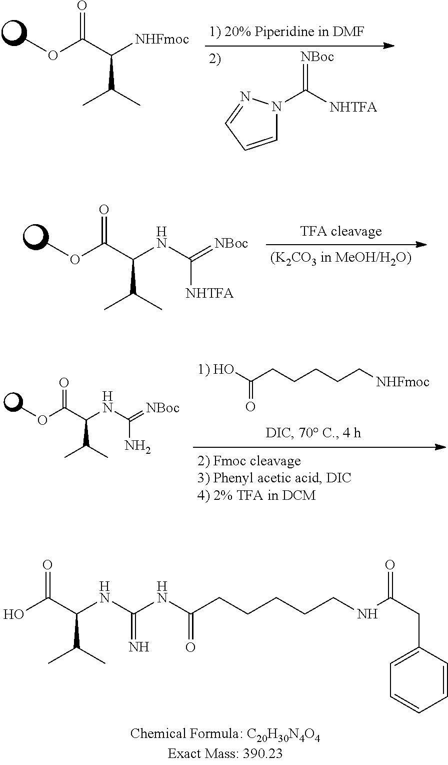

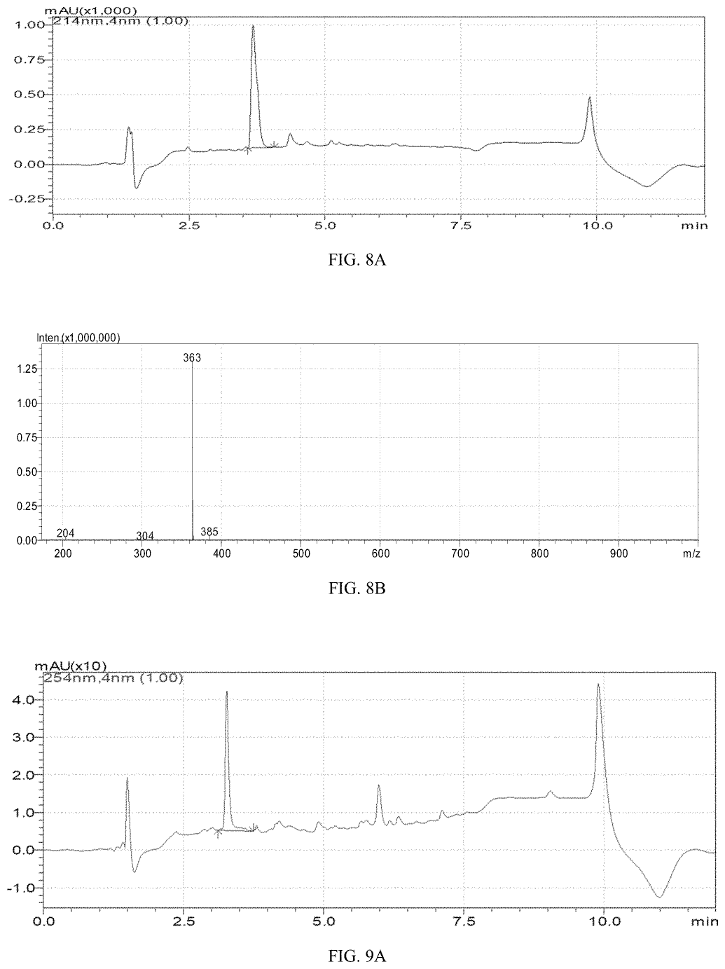

Valine-Gua-N-Me-Phenylalanine-Acetic Anhydride (FIGS. 8A and 8B) can be obtained from valine, N-methyl phenylalanine and acetic anhydride as shown in Scheme 5 below:

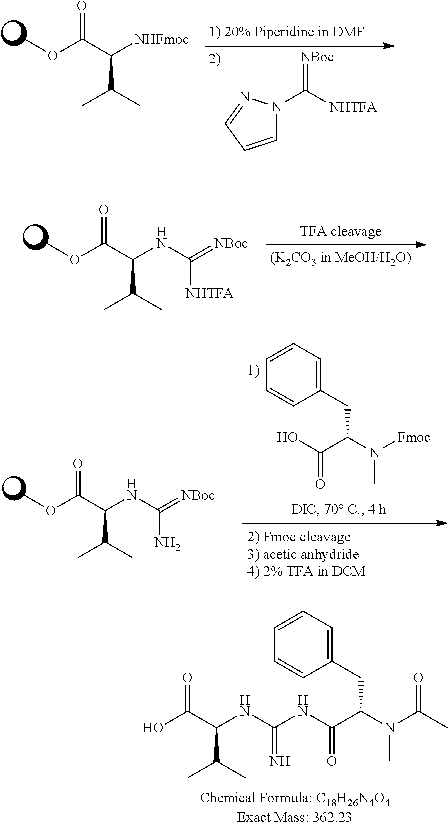

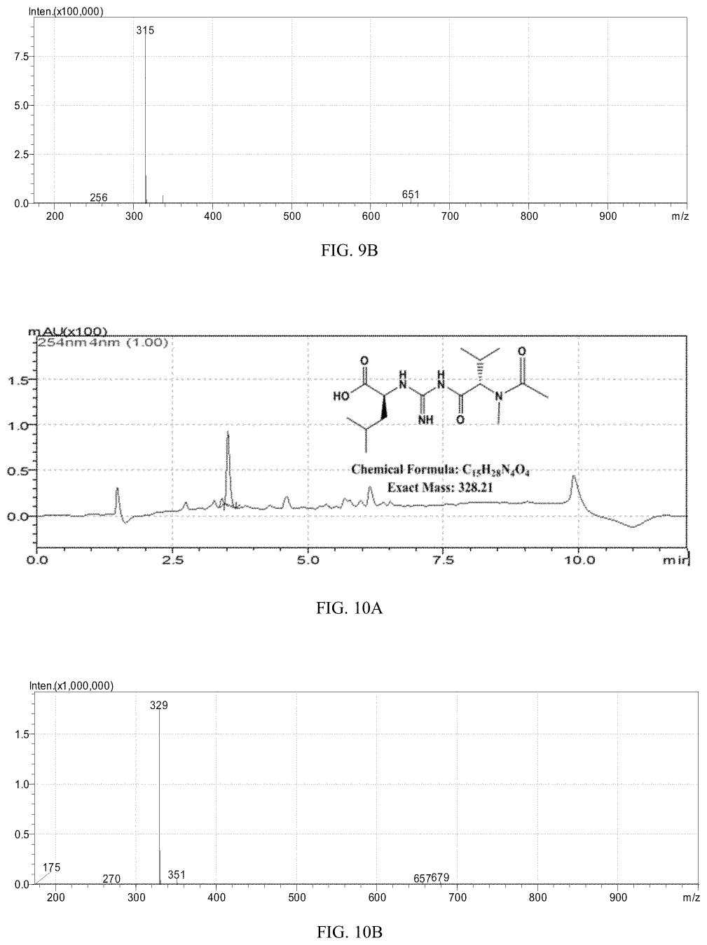

Valine-Gua-N′Me-valine-acetic anhydride (FIGS. 9A and 9B) can be obtained from valine, N-methyl valine and acetic anhydride as shown in Scheme 6 below:

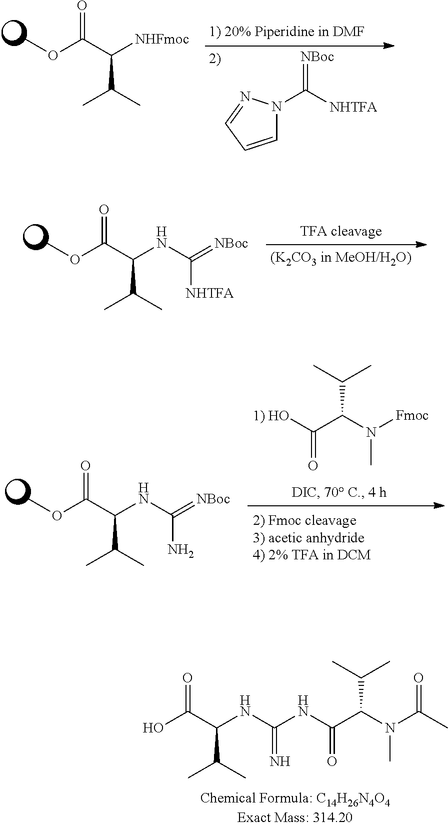

Leucine-Gua-N-Me-Valine-Acetic Anhydride (FIGS. 10A and 10B) can be obtained from leucine, N-methyl valine and acetic anhydride as shown in Scheme 7 below:

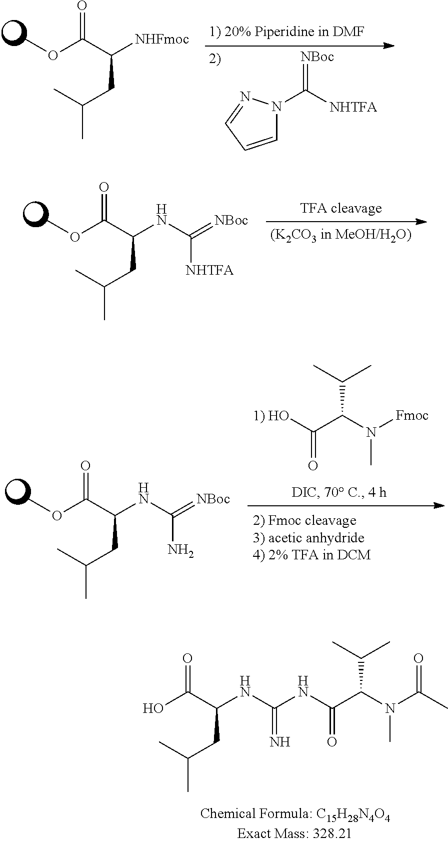

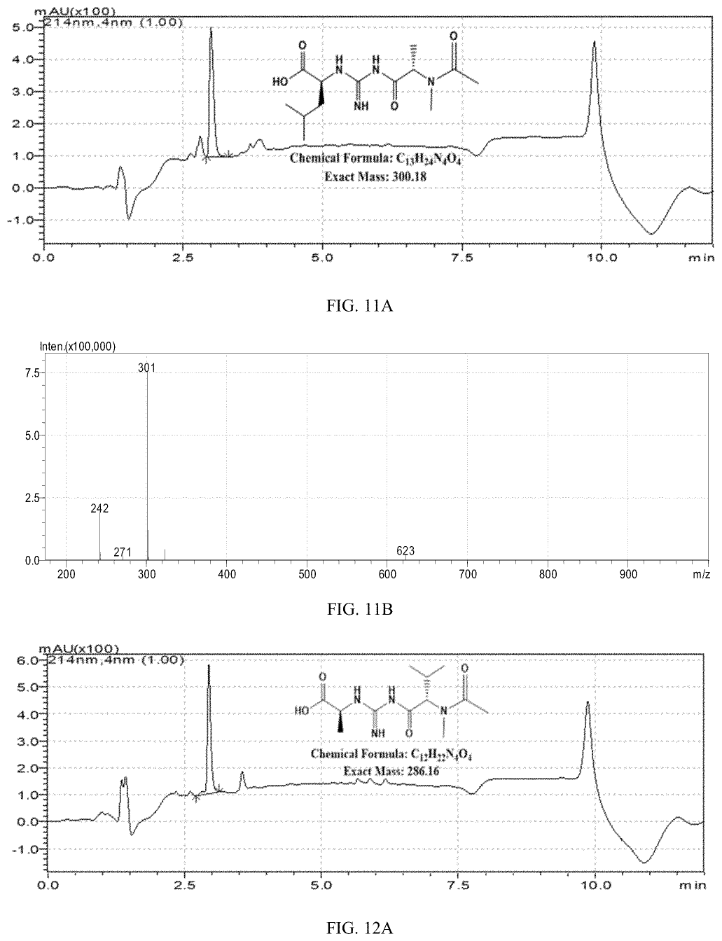

Leucine-Gua-N-Me-Alanine-Acetic Anhydride (FIGS. 11A and 11B) can be obtained from leucine, N-methyl alanine and acetic anhydride as shown in Scheme 8 below:

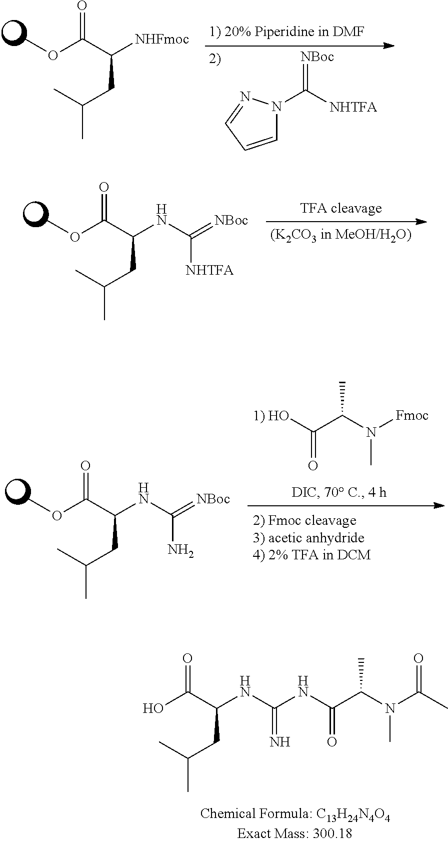

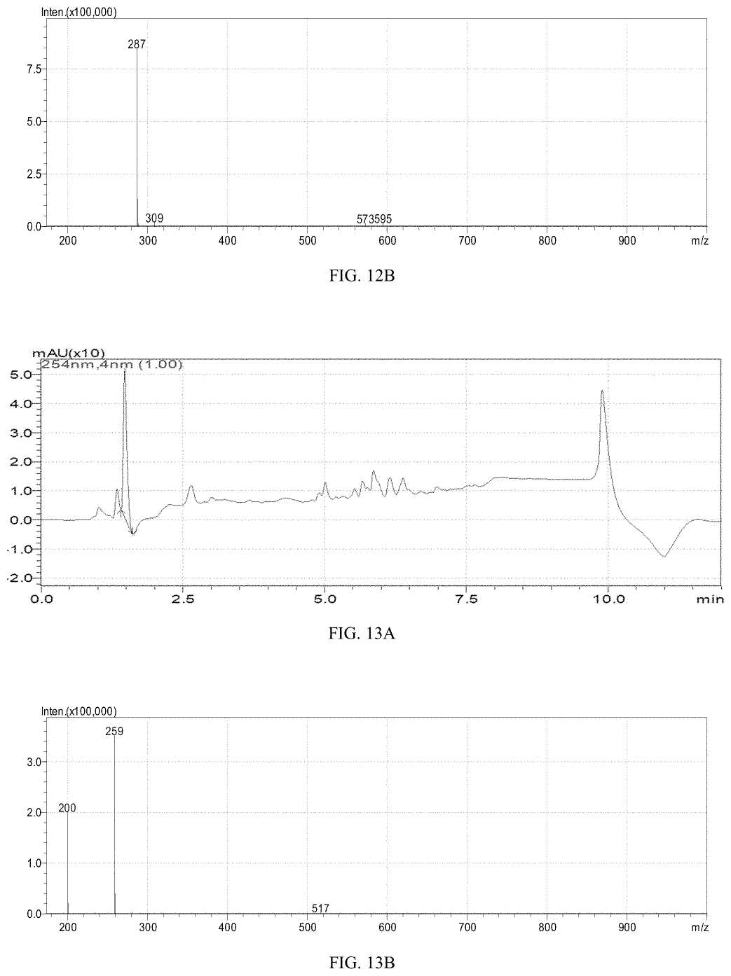

Alanine-Gua-N′Me-Valine-Acetic Anhydride (FIGS. 12A and 12B) can be obtained from alanine, N-methyl valine and acetic anhydride as shown in Scheme 9 below:

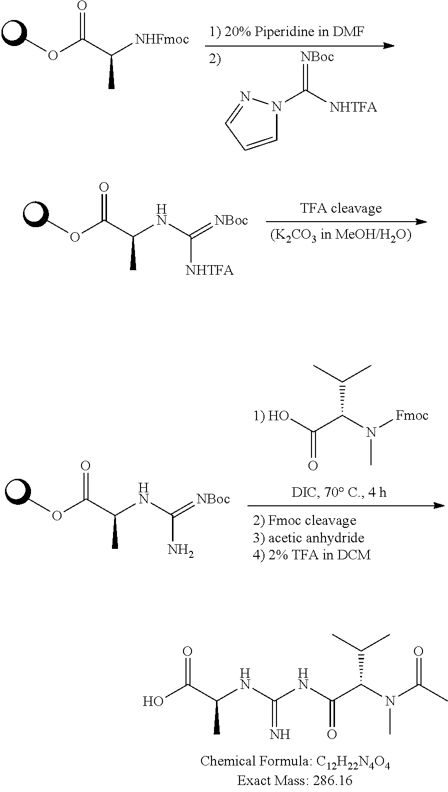

Alanine-Gua-N-Me-Alanine-Acetic Anhydride (FIGS. 13A and 13B) can be obtained from alanine, N-methyl alanine and acetic anhydride as shown in Scheme 10 below:

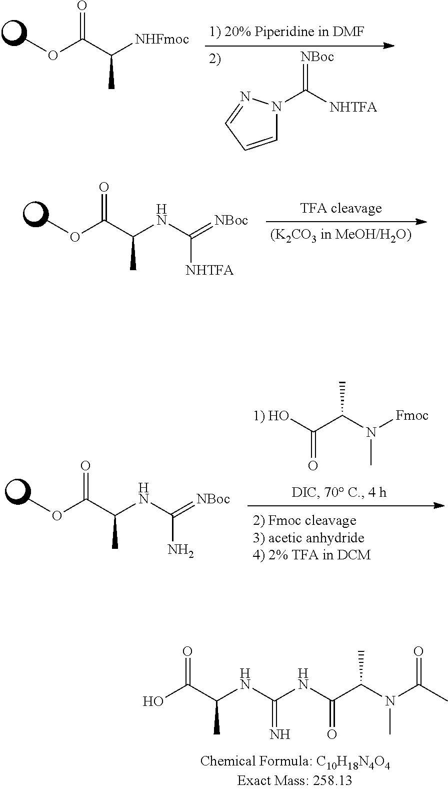

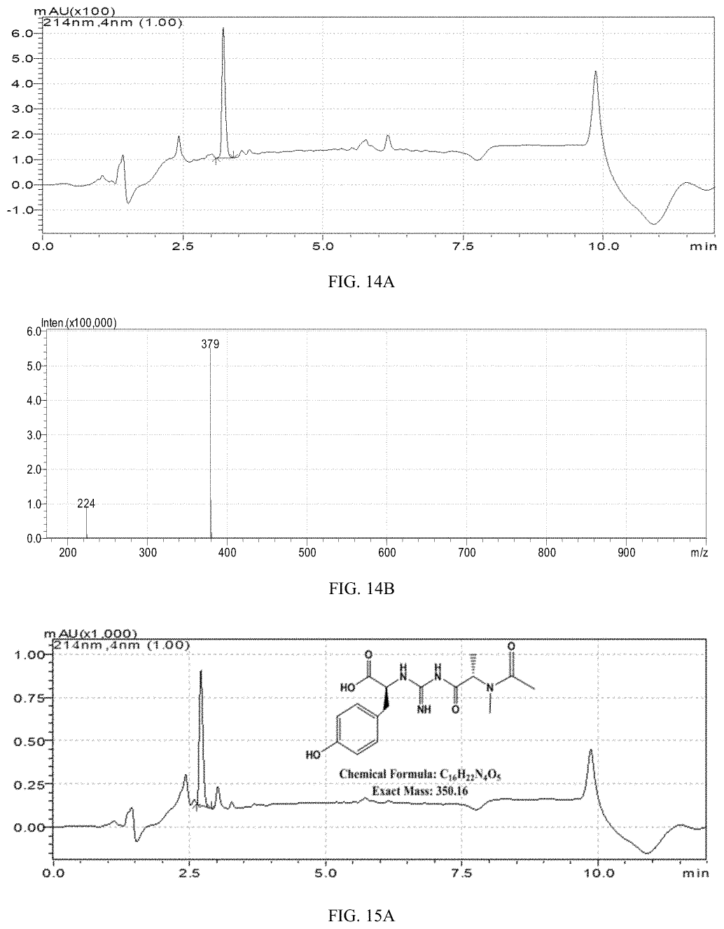

Tyrosine-Gua-N-Me-Valine-Acetic Anhydride (FIGS. 14A and 14B) can be obtained from tyrosine, N-methyl valine and acetic anhydride as shown in Scheme 11 below:

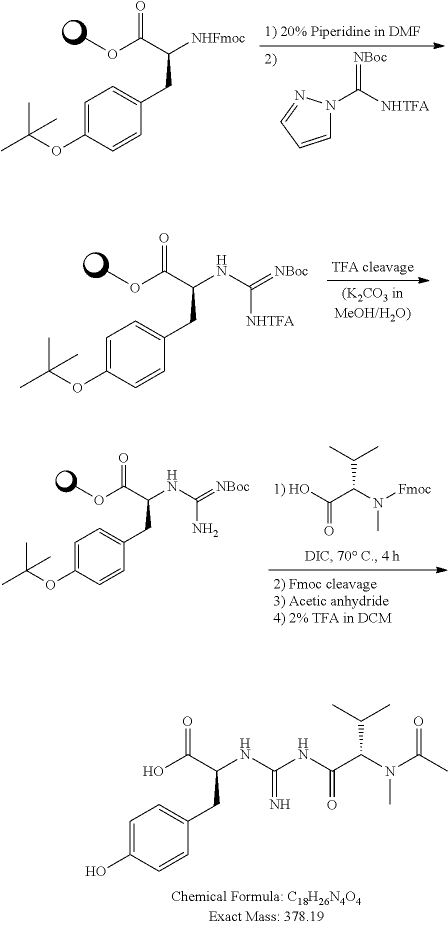

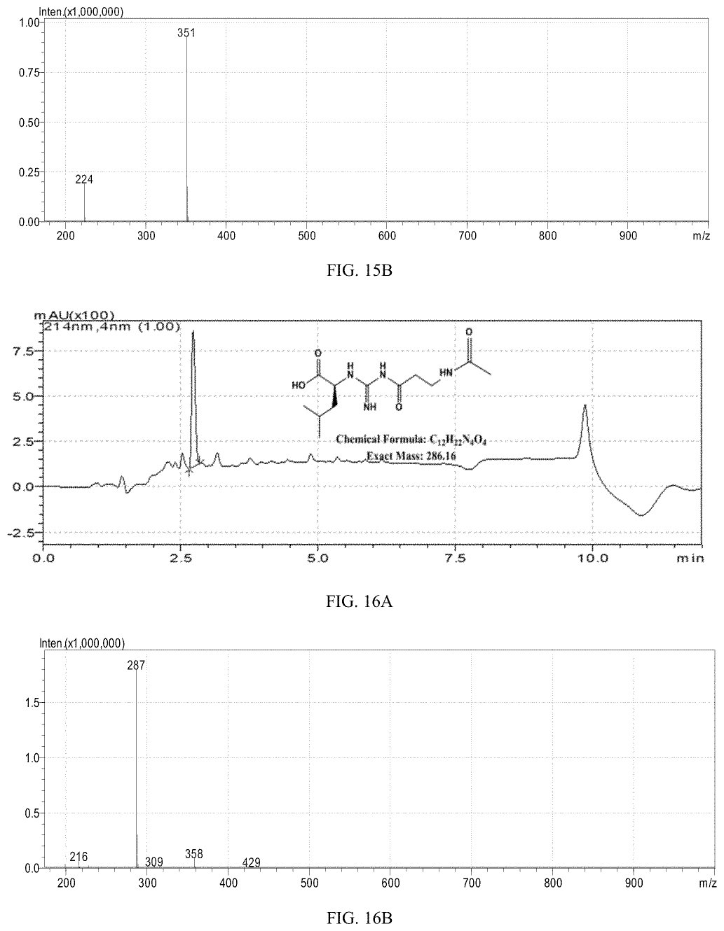

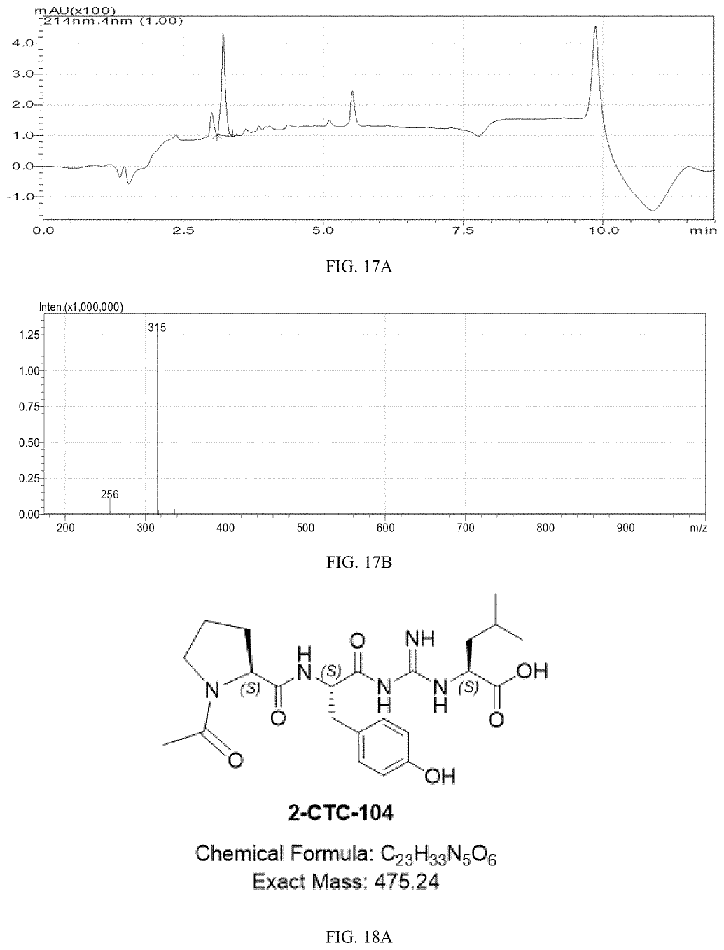

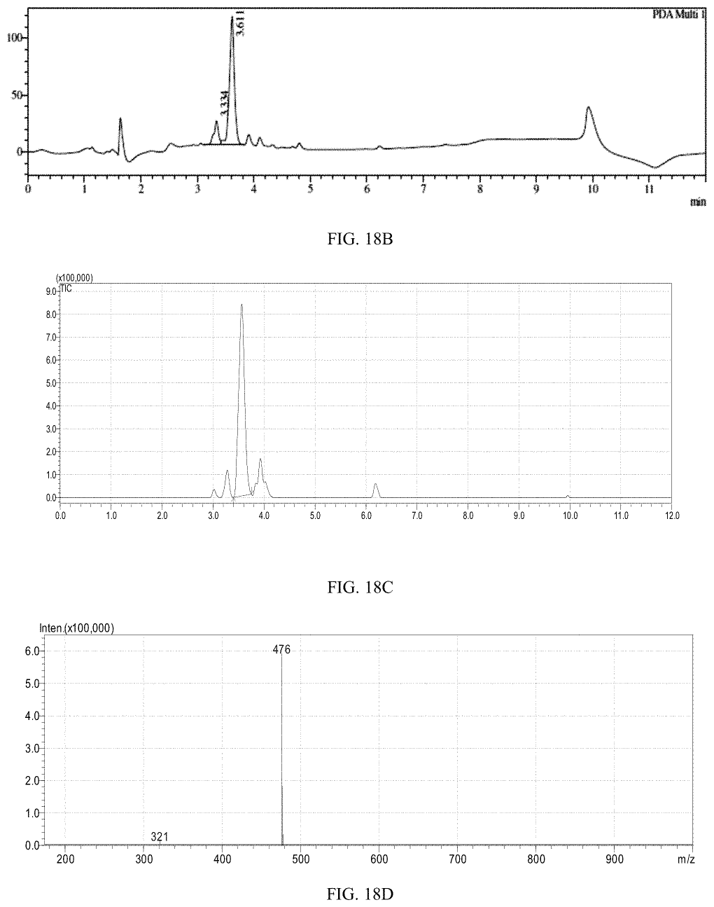

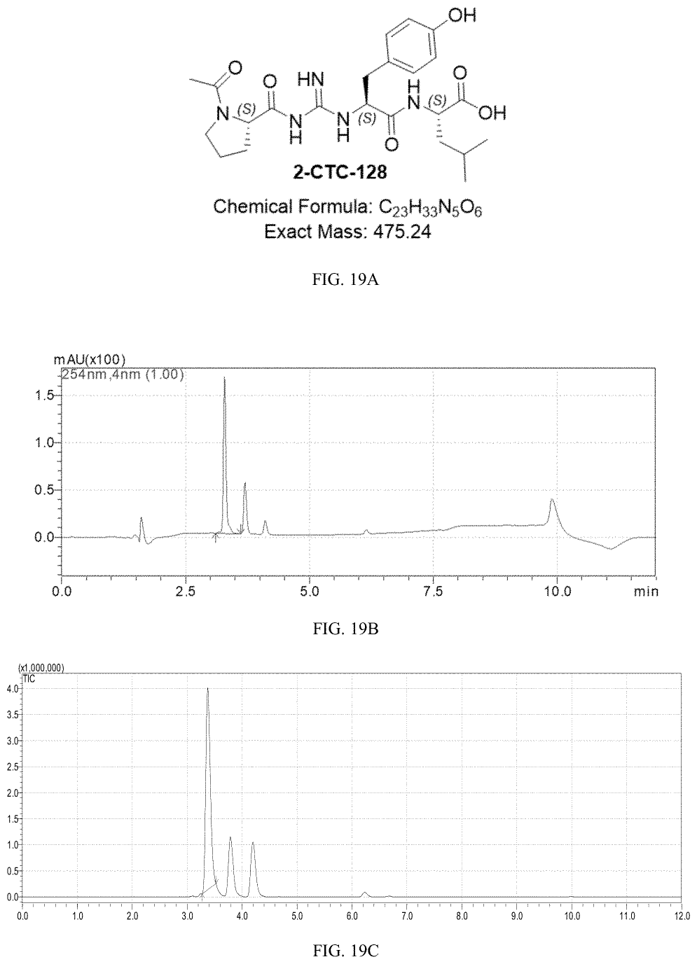

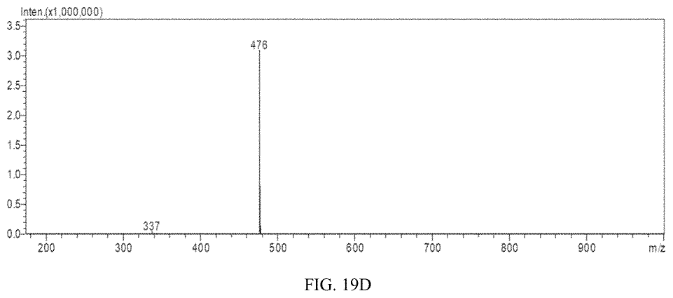

Tyrosine-Gua-N′Me-Alanine-Acetic Anhydride (FIGS. 15A and 15B) can be obtained from tyrosine, N-methyl alanine and acetic anhydride as shown in Scheme 12 below:

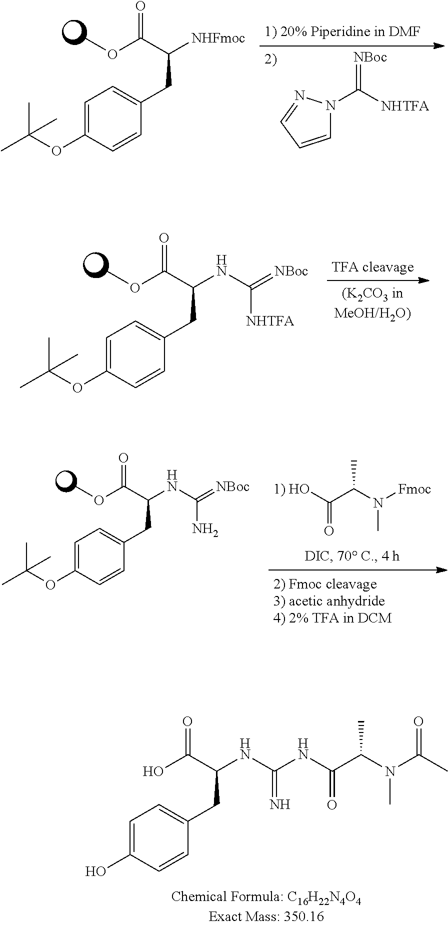

Leucine-Gua-β-Alanine-Acetic Anhydride (FIGS. 16A and 16B) can be obtained from leucine, beta alanine and acetic anhydride as shown in Scheme 13 below:

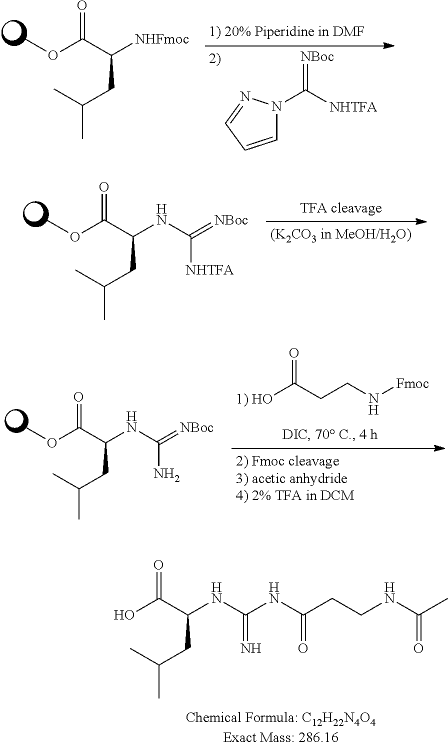

Valine-Gua-Leucine-Acetic Anhydride (FIGS. 17A and 17B) can be obtained from valine, leucine and acetic anhydride as shown in Scheme 14 below:

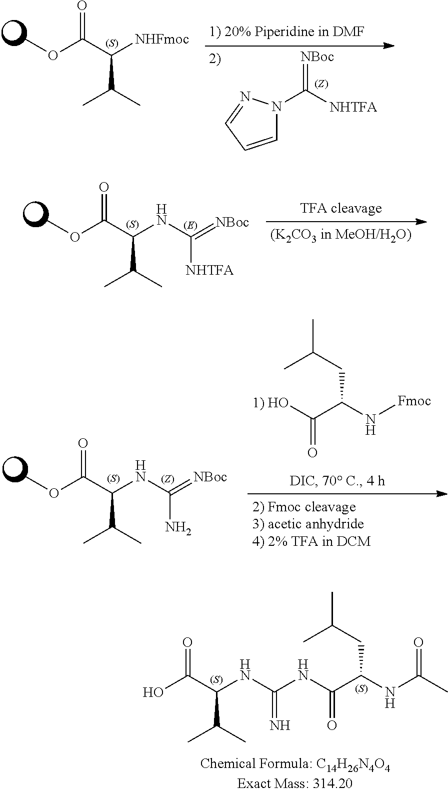

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A peptidomimetic having a general structure of: 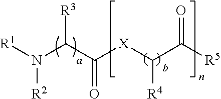
wherein
a≥1; b≥1; and n≥1;
R¹ and R⁵ are each selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —C(═NR^{b})—NR^{c}R^{b}, —OR^{d} and hydroxylalkyl, wherein R^{a}, R^{c}, R^{b} , and R^{d} are each independently selected from hydrogen, —NH₂, alkyl, substituted alkyl, cycloalkyl, substituted cycloalkyl, heterocycloalkyl, substituted heterocycloalkyl, haloalkyl, and acyl;
R² is selected from hydrogen, alkyl, substituted alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl;
each R³ is selected from amino acid side chains and can be present or absent, or R² and R³ taken together with the nitrogen atom and carbon atom to which they are connected, form a substituted 3- to 8-membered heterocyclic ring when a=1;
each R⁴ is selected from side chains of natural amino acids and can be present or absent, or R⁴ on the alpha or beta position and the adjacent R⁶ taken together with the nitrogen atom and carbon atom to which they are connected, form a substituted or unsubstituted 3- to 8-membered heterocyclic ring;
each X is independently selected from —NR⁶—and —NR⁶—C(═NR⁶)—NR⁶—, and at least one X is —NR⁶—C(═NR⁶)—NR⁶,
and each R⁶ is selected from hydrogen, alkyl, aryl, substituted aryl, heteroalkyl, substituted heteroalkyl, heteroaryl, substituted heteroaryl, cycloalkyl, substituted cycloalkyl, heterocycloalkyl, substituted heterocycloalkyl, cycloalkenyl, substituted cycloalkenyl, alkenyl substituted alkenyl, alkynyl, haloalkyl, acyl, substituted acyl, —SR^{a}, —NR^{c}R^{b}, —OR^{d} and hydroxylalkyl.

2. The peptidominetic of claim 1, a and each b being 1-10; and n being 1-20.

3. The peptidominetic of claim 1, R¹ being hydrogen, alkyl, acyl, or guanidine.

4. The peptidominetic of claim 1, R² being hydrogen or alkyl.

5. The peptidominetic of claim 1, wherein R⁵ is —SR^{a}, —NR^{c}R^{b}, or —OR^{d} , wherein R^{a}, R^{c}, R^{b}, and R^{d} are each independently selected from hydrogen, —NH₂, alkyl, substituted alkyl, cycloalkyl, substituted cycloalkyl, heterocycloalkyl, substituted heterocycloalkyl, haloalkyl, and acyl.

6. The peptidominetic of claim 1, wherein the peptidomimetic has a structure of: 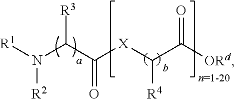
wherein each X is independently selected from —NH— and —NH—C(═NH)—NH—, and at least one X is —NH—C(═NH)—NH—.

7. The peptidominetic of claim 1, wherein the peptidomimetic has a structure of:

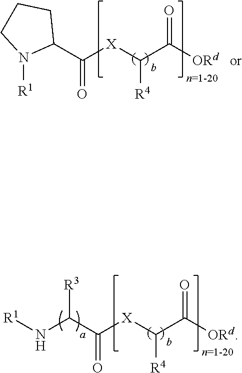

8. The peptidominetic of claim 1, wherein peptidomimetic is selected from

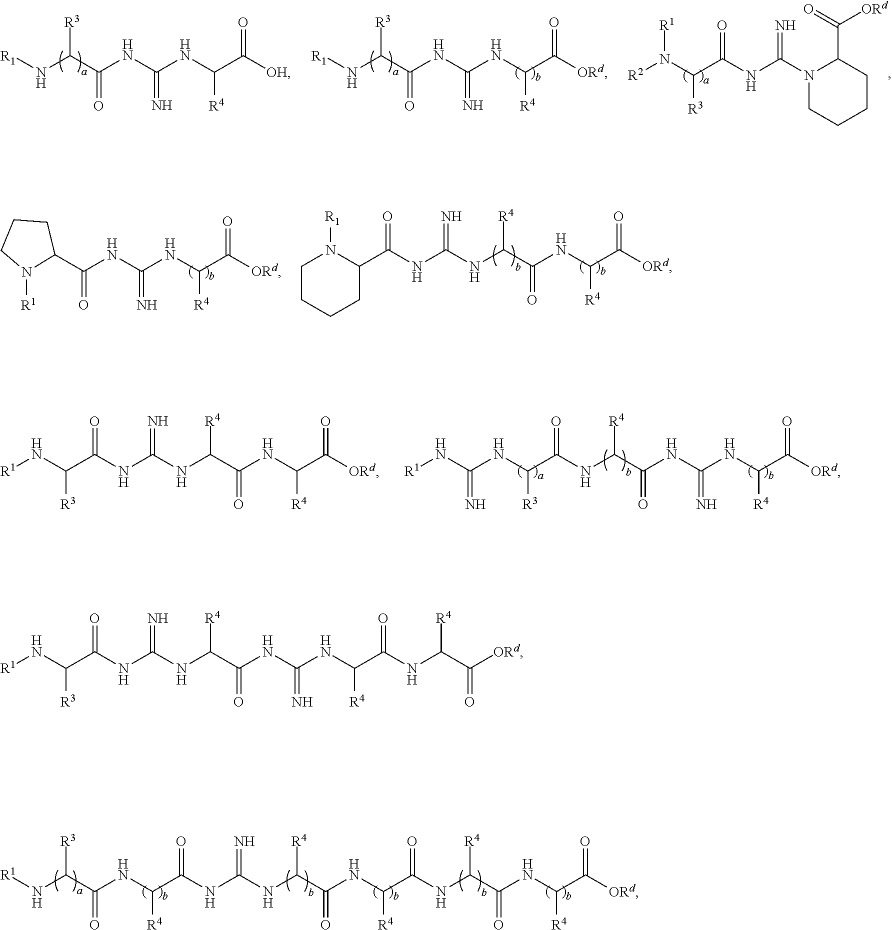

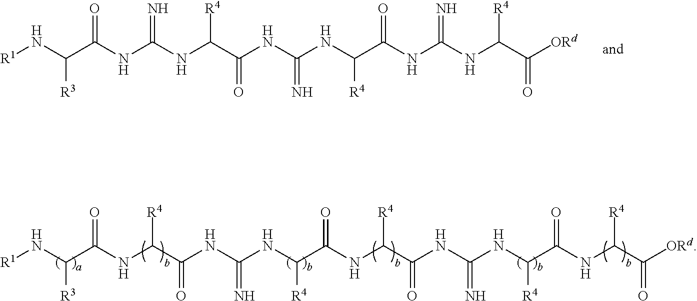

9. The peptidominetic of claim 1, wherein peptidomimetic is selected from

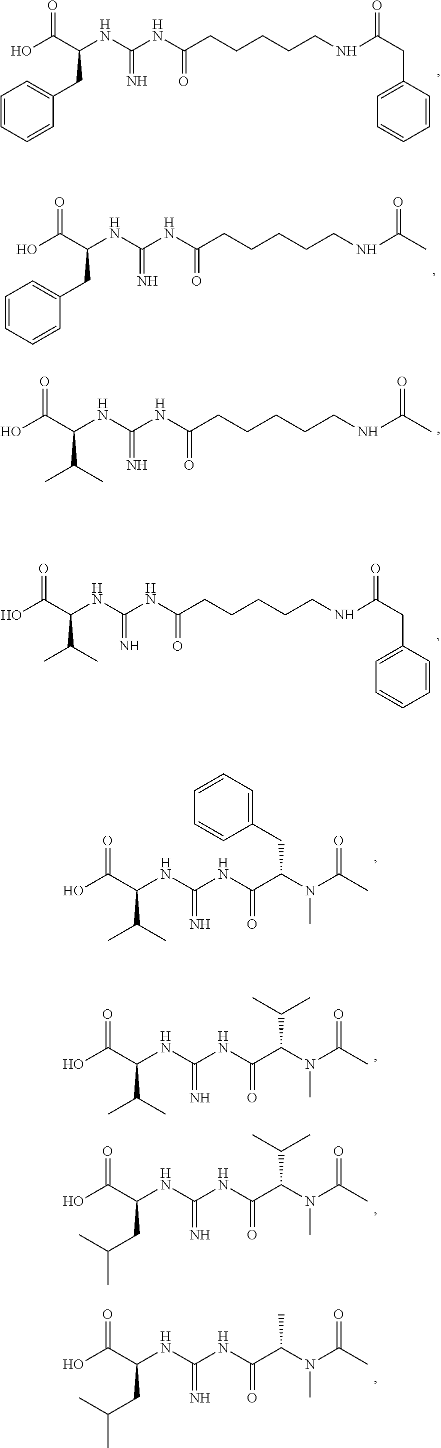

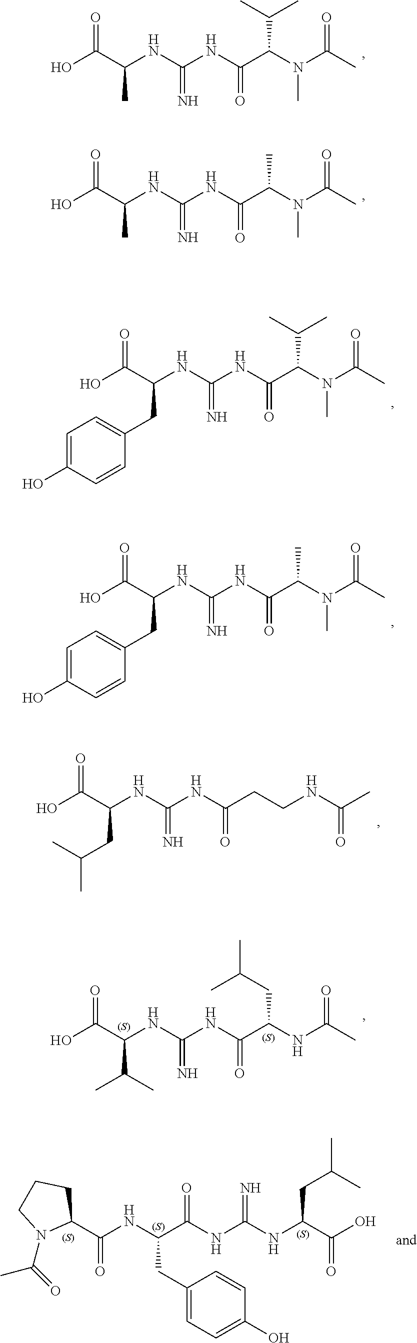

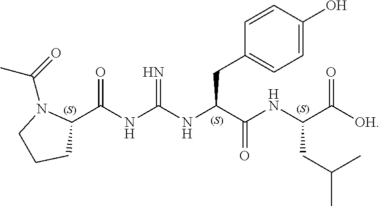

10. A method for synthesizing a peptidomimetic comprising one or more guanidino acids, the method comprising:
1) contacting an amino acid with a guanilidation reagent to form a protected guanidino acid that comprises a guanidine with one or more protection groups;
2) removing the one or more protection groups of the protected guanidino acid;
3) mixing the deprotected guanidino acid with a pre-protected amino acid to couple the pre-protected amino acid to the guanidine of the guanidino acid;
4) removing the protection of the product of step 3);
5) optionally, treating the product of step 4) with the guanilidation reagent; and
6) repeating steps 2) to 4), or 3) to 4).

11. The method of claim 10, the amino acid being a natural or non-natural, modified or non-modified amino acid.

12. The method of claim 10, the guanilidation reagent having a structure of 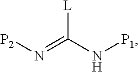
wherein L is a leaving group selected from halogen, —SMe, pyrazole, substituted pyrazole, and ammonium; P1 and P2 are orthogonal protecting groups each independently selected from fluorenylmethoxycarbonyl (Fmoc), tert-butyloxycarbonyl (Boc), benzyl chloroformate (Cbz), acetyl (Ac), trifluoroacetyl (TFA), phthalimide, benzyl (Bn), trityl (Trt), benzylideneamine, and tosyl (Ts).

13. The method of claim 10, the guanilidation reagent being selected from N-Boc-N′-TFA-pyrazole-1-carboxamidine 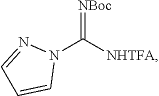
1,3-Bis(tert-butoxycarbonyl)-2-methyl-2-thiopseudourea 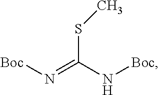 [SMILES: CS/C(=N\C(=O)OC(C)(C)C)NC(=O)OC(C)(C)C]
and 1,3 -Bis(tert-butoxycarbonyl)guanidine

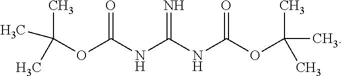 [SMILES: CC(C)(C)OC(=O)NC(=N)NC(=O)OC(C)(C)C]

14. The method of claim 10, the protected amino acid comprising a protection group selected from for example, fluorenylmethoxycarbonyl (Fmoc), tert-butyloxycarbonyl (Boc), benzyl chloroformate (Cbz), acetyl (Ac), trifluoroacetyl (TFA), phthalimide, benzyl (Bn), trityl (Trt), benzylideneamine, and tosyl (Ts).

15. (canceled)

16. A method for synthesizing a peptidomimetic, the method comprising:
preparing one or more pre-protected guanidino acids comprising one or more protection groups at the guanidine;
coupling the one or more pre-protected guanidino acids in a stepwise manner in the presence of a coupling reagent; and
removing the one or more protection groups.

17. The method of claim 16, preparing one or more pre-protected guanidino acids comprising contacting an amino acid with a guanilidation reagent, the amino acid being natural or non-natural, modified or non-modified amino acids.

18. The method of claim 17, the guanilidation reagent having a structure of 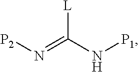
wherein L is a leaving group selected from halogen, —SMe, pyrazole, substituted pyrazole, and ammonium; P1 and P2 are orthogonal protecting groups each independently selected from fluorenylmethoxycarbonyl (Fmoc), tert-butyloxycarbonyl (Boc), benzyl chloroformate (Cbz), acetyl (Ac), trifluoroacetyl (TFA), phthalimide, benzyl (Bn), trityl (Trt), benzylideneamine, and tosyl (Ts).

19. The method of claim 17, the guanilidation reagent being selected from N-Boc-N′-TFA-pyrazole-1-carboxamidine 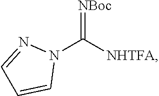
1,3-B is(tert-butoxycarbonyl)-2-methyl-2-thiopseudourea 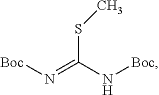 [SMILES: CS/C(=N\C(=O)OC(C)(C)C)NC(=O)OC(C)(C)C]
and 1,3-Bis(tert-butoxycarbonyl)guanidine

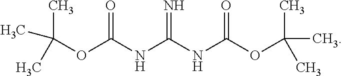 [SMILES: CC(C)(C)OC(=O)NC(=N)NC(=O)OC(C)(C)C]

20. The method of claim 16, the coupling reagent being selected from N,N-disubstituted carbodiimides, acyl chloride, acyl fluoride, anhydrides, BOP, PyBOP, PyAOP, PyOxim, TOTU, TSTU, COMU, DEPBT, HBTU, HATU, PyAOP, and HCTU.

21. The peptidominetic of claim 1, wherein R¹ is hydrogen; R² is hydrogen; R³ is an amino acid side chain; a is 1; n is 3; b is 1; each X is —NR⁶—C(═NR⁶)—NR⁶—, wherein R⁶ is hydrogen; each R⁴ is a side chain of natural amino acids; and R⁵ is —OR^{d}, wherein R^{d} is hydrogen.
